(19) United States
(12) Patent Application Publication
(10) Pub. No.: US 2026/0148834 A1
(43) Pub. Date: May 28, 2026

(54) AI-Driven Emotional Intelligence App

(71) Applicants: Eduardo Enrique Rivodo Zambrano, Miami, FL (US); Maria Elena Garassini Chavez, Miami, FL (US); Maria Alejandra del Carmen Rivodo Rojas, Miami, FL (US)

(72) Inventors: Eduardo Enrique Rivodo Zambrano, Miami, FL (US); Maria Elena Garassini Chavez, Miami, FL (US); Maria Alejandra del Carmen Rivodo Rojas, Miami, FL (US)

(21) Appl. No.: 18/963,401

(22) Filed: Nov. 27, 2024

(51) Int. Cl. G16H 20/70; G16H 50/20

(52) U.S. Cl. CPC G16H 20/70; G16H 50/20

(57) ABSTRACT

A computer-implemented method for managing user emotions using artificial intelligence includes receiving emotional state input from a user; processing the emotional state input with an artificial intelligence psychologist code with a Retrieval-Augmented Generation (RAG) code and a custom Large Language Model (LLM); retrieving information from a knowledge base containing documents related to anxiety management and user-specific information; generating personalized emotional management recommendations based on the processed emotional state input and retrieved information; providing the personalized emotional management recommendations to the user; and tracking user behavior and engagement with the provided recommendations.

### BACKGROUND OF THE INVENTION

For thousands of years, people have been trying to understand how emotions occur, what they mean, and how they affect people. This ongoing curiosity highlights the need for tools that can help individuals navigate their emotional landscape. As our understanding of mental health and emotional intelligence has evolved, it has become clear that traditional one-size-fits-all approaches are inadequate for addressing the varied emotional needs of individuals, particularly in the workplace.

In recent decades, there has been a growing recognition of emotional intelligence (EQ) as distinct from traditional intelligence (IQ). This shift has created a demand for resources that can help people develop and balance their emotional intelligence. Within this spectrum lies a growing interest in understanding and managing human emotions, which play a critical role in human interaction, decision-making, and overall mental health. Traditional methods for monitoring and influencing emotional states have relied primarily on direct human intervention, such as psychological therapy or peer support. However, such methods are often limited by the availability of professionals, subjective biases, and the general challenge of articulating nuanced emotional experiences.

### SUMMARY OF THE INVENTION

In one aspect, a computer-implemented method for managing user emotions using artificial intelligence includes receiving emotional state input from a user; processing the emotional state input with an artificial intelligence psychologist code with a Retrieval-Augmented Generation (RAG) code and a custom Large Language Model (LLM); retrieving information from a knowledge base containing documents related to anxiety management and user-specific information; generating personalized emotional management recommendations based on the processed emotional state input and retrieved information; providing the personalized emotional management recommendations to the user; and tracking user behavior and engagement with the provided recommendations.

In another aspect, the method includes a computer-implemented process for emotion management aided by AI, which involves obtaining user emotional input, analyzing it through an AI psychologist module with a RAG and an LLM, sourcing pertinent data from a knowledge repository focused on anxiety management and specific user details, formulating tailored guidance for emotion control based on this analysis and information, and presenting these recommendations to the user in the form of reports, self-care activities, progress tracking and insights. Additionally, the method tracks the user's interaction and adherence to the recommendations using analytics.

Advantages of one implementation may include one or more of the following. In one implementation at Emotionall.co, the AI-powered application offers a comprehensive approach to enhancing users' emotional well-being and interpersonal skills by focusing on several key areas. The app aims to improve mood balance by increasing positive emotions while reducing negative ones, fostering greater self-awareness to help users better understand and recognize their own emotional states. Emotionall also emphasizes the development of resilience, equipping users with the tools to effectively navigate life's challenges and stressors. By enhancing social skills, the app promises to improve users' ability to communicate and form meaningful relationships. The platform's scientific approach ensures accurate measurement of progress, utilizing research-based methodologies to quantify improvements in emotional intelligence. Emotionall's unique perspective on emotion control encourages users to view emotions as actionable alerts rather than persistent mood states, promoting a more proactive approach to emotional management. Additionally, the app focuses on boosting motivation, which can lead to significant improvements in both personal and professional spheres. Through this multifaceted approach, Emotionall strives to provide users with a powerful tool for comprehensive emotional growth, self-improvement, and the cultivation of more fulfilling interpersonal relationships, ultimately contributing to an enhanced overall sense of well-being and life satisfaction. Other advantages may include one or more of the following:

Personalized Emotion Management: The AI system can tailor its responses and strategies to cater to individual emotional states and personal preferences, thus offering a high level of customization in emotion management.

Continuous Availability: Unlike human professionals who may not be available around the clock, this system would provide support at any time, making emotional assistance accessible to users whenever needed.

Data-Driven Insights: By drawing on extensive knowledge repositories and leveraging machine learning, the system can offer informed strategies for emotion regulation based on the latest research and evidence from the field of psychology.

Proactive Engagement: The system may include features designed to engage with users proactively, rather than waiting for them to initiate contact. This can help in preemptively managing emotional states.

Non-Invasive Intervention: The AI-driven method offers a non-invasive alternative to pharmaceutical interventions for emotional regulation, potentially reducing the reliance on medication and its associated side effects.

Cost-Effective: Automating the process of emotion regulation can significantly decrease the costs associated with traditional therapy, making it a more affordable option for users.

Real-Time Adjustment: The AI system can respond in real-time to changes in emotional state, making its interventions more relevant and timely.

Enhanced Privacy: Users may feel more comfortable interacting with an AI system when discussing sensitive emotional issues, thus potentially leading to more honest and open communication.

Increased Reach: The system can be scaled to serve a large number of users simultaneously, which can significantly extend the reach of mental health support, particularly in underserved areas.

Improved Mental Health Outcomes: By providing consistent, accessible, and tailored support, one implementation could improve mental health outcomes for many individuals, leading to better overall well-being.

Extensive Data Collection: The system can collect a large amount of data on emotional responses and intervention outcomes, which can be used to improve the system itself and contribute to scientific research.

By integrating these various aspects of AI and machine learning with emotional intelligence, the system represents a significant advance in the field of affective computing and the broader objective of promoting psychological well-being. With burnout becoming a significant issue affecting both individual well-being and organizational effectiveness, there is a pressing need for solutions that can help identify, address, and overcome emotional exhaustion in professional settings. Given that mental health strategies should be as varied as individuals themselves, the app addresses the need for customized emotional management tools that can adapt to each user's unique emotional profile and needs. The platform enables individuals to better comprehend and manage their emotional responses in various situation and provides a comprehensive, personalized approach to understanding and improving user emotional intelligence in both personal and professional contexts.

### BRIEF DESCRIPTION OF DRAWINGS

FIG. 1A shows a flowchart of an exemplary AI-driven emotion management process for personalized psychological support.

FIG. 1B shows an exemplary process to respond to a mental health request.

FIG. 2A shows an exemplary AI-driven emotion management system for personalized psychological support.

FIG. 2B shows more details one implementation of FIG. 2A.

FIG. 3 illustrates an example of a response to a request for emotional support.

FIG. 4A shows an exemplary mobile app portion of the AI-driven emotion management system for personalized psychological support.

FIG. 4B shows an exemplary cloud-based portion of the AI-driven emotion management system for personalized psychological support.

FIG. 5 shows an exemplary emotional survey process.

FIGS. 6A-6L show exemplary user interface layouts for the emotional surveys.

FIG. 7 shows an exemplary process to collect daily survey data.

FIGS. 8A-8W show exemplary user interface layouts for the daily surveys.

FIGS. 9-10 show an exemplary process to collect and to create dynamic survey data.

FIG. 11A-11I show exemplary user interface layouts for the dynamic surveys.

FIG. 12 shows an exemplary process to perform Emotional Survey: User Emotional Placement-Customized Emotion Management Report

FIG. 13 shows an exemplary process to perform Activity Ranking and generate Personalized Self-Care Recommendations

FIG. 14 shows an exemplary process to perform Progress Tracking, Evaluating and Visualizing User Emotions.

FIG. 15 shows an exemplary process to perform Daily Survey for emotions.

FIG. 16 shows an exemplary process to perform Insights Report Generation by Aggregating, Analyzing, and Delivering Emotional Trends.

### DETAILED DESCRIPTION OF THE INVENTION

One implementation relates to a computer-implemented method for personal emotion regulation, which is facilitated by an artificially intelligent entity designed to operate as a psychological aid for users. Through the steps described, the computer-implemented method operationalizes an advanced AI system capable of delivering individualized emotional management support, reflecting an intuitive understanding of human emotions and a sophisticated capability for adaptive learning and personalized care in managing user emotions such as anxiety, among others.

The system applies several computer performance improvements and technological advancements to enhance the system's functionality and efficiency. AI-Driven Emotion Recognition algorithms are used to analyze facial expressions, voice intonations, and text inputs in real-time. This AI-driven approach enables one or more of: rapid and accurate emotion detection; processing of complex emotional data with minimal latency; continuous learning and improvement of the emotion recognition model. The system integrates multiple data streams simultaneously, including: visual data from facial expressions, audio data from voice analysis, and textual data from user inputs and the multi-modal approach allows for more comprehensive and accurate emotional analysis compared to single-mode systems. The app implements a sophisticated real-time feedback mechanism that: processes user interactions, provides immediate emotional insights, and adapts recommendations based on user responses. This continuous feedback loop enhances the system's responsiveness and personalization capabilities. The app leverages a scalable cloud-based infrastructure that allows for: dynamic resource allocation based on user demand; improved processing speed during peak usage periods; seamless updates and feature rollouts without service interruption. The app utilizes personalized machine learning models that: adapt to individual user patterns over time, improve prediction accuracy for emotional states, and reduce computational load by focusing on relevant data for each user. These technological improvements collectively enhance the app's performance, enabling it to process complex emotional data efficiently, provide personalized insights in real-time, and continuously improve its accuracy and effectiveness. This sophisticated implementation goes beyond generic computer applications, addressing specific challenges in emotional intelligence technology and demonstrating tangible advancements in computer-related functionality.

The method commences at step S100 by receiving emotional state input from a user, which includes qualitative and quantitative data indicative of the user's current emotional state. This input can be actively provided by the user or passively collected through sensors or user interaction data. Once acquired, this emotional state input serves as the foundational data from which the system commences analysis and subsequent emotion regulation guidance.

One implementation encompasses a method for managing emotions with an artificial intelligence (AI) system, where at step S102, the emotional state input from a user is processed by a specialized AI psychologist module. This module integrates a Retrieval-Augmented Generation (RAG) component with a bespoke Large Language Model (LLM), both of which work in concert to interpret and analyze the emotional input. The RAG module uses its retrieval capabilities to fetch relevant context and information, which informs the understanding and processing carried out by the LLM. The LLM—custom-designed for this application-leverages its extensive language understanding to discern the nuances of the emotional state conveyed by the user's input. The combination of these two elements establishes a robust framework for accurately deciphering the emotional indicators provided by the user, thus laying a cognitive foundation for subsequent steps in the emotion management method.

Upon receiving an emotional state input from a user, the AI psychologist module commences its operation by processing the input (S102). This involves deciphering the various nuances of the user's emotional state through advanced algorithmic interpretations. Once the emotional state input has been thoroughly analyzed, the AI psychologist module moves on to the next critical function as marked by reference label S104.

Reference label S104 denotes a pivotal step in the method: the retrieval of relevant information from a meticulously curated knowledge base. This knowledge base is not merely a static collection of generic anxiety management documents; it is a dynamic repository that also encompasses data specific to the individual user. By engaging in this retrieval process, the AI psychologist module ensures that the emotional management recommendations it generates are informed by a wide array of pertinent information, ranging from established psychological strategies to particular insights regarding the user's personal emotional patterns and history.

Following the retrieval of information, the AI psychologist module synthesizes the input and the curated data to generate personalized emotional management recommendations (S106). These recommendations are designed to be responsive to the user's unique needs and preferences in managing their emotional state. The subsequent step involves the presentation of these personalized recommendations to the user through a mobile application interface (S108), offering an accessible and user-friendly medium for emotional support.

Additionally, the method comprises an analytics component that diligently tracks the user's interaction with, and adherence to, the emotional management recommendations provided (S110). This tracking capability not only offers insights into the user's engagement but also contributes to refining the effectiveness of the assistance rendered by the AI psychologist module.

The generation of these personalized recommendations is informed by the integrated Retrieval-Augmented Generation (RAG) module and a tailored Large Language Model (LLM) within the AI psychologist module. The RAG module assists in dynamically sourcing and incorporating pertinent external data from the knowledge base, while the custom LLM interprets the user's emotional input in a manner that simulates the cognitive processes of a professional psychologist. Together, these components of the AI psychologist module work in synergy to conceptualize and produce specific, actionable, and individualized advisories aimed at assisting users in navigating and regulating their emotions (S106). The system provides a refined analysis that captures the nuances of the user's emotional state, thereby facilitating the generation of recommendations that resonate on a deeply personal level with the user.

Following the processing step, the AI psychologist module retrieves additional relevant information from a comprehensive knowledge base. This knowledge base contains an array of documents related to anxiety management along with user-specific information which may include past entries, user profiles, and historical behavioral patterns. The retrieval of this information is crucial as it allows the AI psychologist module to contextualize the user's emotional state within the broader spectrum of their emotional history and available psychological resources.

Finally, user behavior and engagement with the provided recommendations are tracked by an analytics module. This module collects and analyzes data related to how the user interacts with the recommendations—such as frequency of use, duration of interaction, user feedback, and any follow-up actions taken by the user. The analytics module serves to measure the efficacy of the recommendations and to facilitate continuous improvement of the system's responses. This feedback loop ensures that the system adapts to the user's changing needs and refines its recommendation algorithms for enhanced personalization over time.

In accordance with one aspect of one implementation, a method and system for providing personalized emotional management recommendations is expanded to further include a feature that enhances user engagement and ensures timely intervention in the emotional regulation process. Once the AI psychologist module has provided personalized emotional management recommendations to the user, there exists an additional step executed by a push notification module within the system. This module is responsible for sending timely updates and reminders to the user. The push notification module operates in consideration of the user's engagement data and preferences to deliver relevant notifications that prompt the user to revisit the recommendations or to engage with new or outstanding tasks aimed at managing their emotional state. The timing and content of these notifications can be intelligently determined based on the user's past behavior, predicted future emotional states, or imminent events that may influence the user's emotional well-being.

For instance, if the analytics module identifies that a user typically engages with the application and its recommendations in the evenings, the push notification module may prioritize sending reminders or updates during that time to increase the likelihood of the user taking actionable steps towards emotional management. Conversely, if a user seems to be neglecting to engage with the system, the push notification module can send prompts at times when the user is most likely to be receptive.

Additionally, the system may allow for user input regarding the scheduling and specific content of push notifications. For example, users may request to receive motivational quotes, mindfulness reminders, or prompts for journaling at specific times of the day. The system is thus configured to receive such preferences and instruct the push notification module to act accordingly.

Upon completion of step S108, wherein personalized emotional management recommendations are provided to the user through a mobile application interface, the system moves to an additional step, which involves the provision of customized self-care activities. These activities are tailored to the individual's specific needs and emotional state, embodying a holistic approach to emotional wellness.

Customized self-care activities may include, but are not limited to, guided meditation exercises, breathing techniques, physical activities such as yoga or walking, or journaling prompts. These activities are selected and generated by the AI psychologist module, which employs the RAG module and LLM to process the user's emotional state input and historical data, as well as information from the extensive knowledge base. By considering factors such as the user's past preferences, current emotional state, and the outcomes of previously recommended activities, the system is capable of curating a set of self-care tasks that are optimally configured to support the user's mental health journey.

For instance, if the user has consistently engaged with recommendations involving physical activity and has provided positive feedback on such recommendations, the system may prioritize suggesting a light physical exercise followed by a guided relaxation technique. Alternatively, if the analytics module detects increased levels of anxiety from the user's input, the system may suggest a journaling exercise that focuses on identifying and addressing the sources of anxiety.

After personalized self-care activities are generated, the system proceeds to present these activities to the user, offering clear instructions and guidance on how to perform them. This is accomplished through a user-friendly interface within the mobile application that allows for easy selection and engagement with the recommended self-care tasks.

Thus, one implementation provides a comprehensive and user-centric system for emotional state management, consisting of both recommendations for emotional management and customized self-care activities, all facilitated through the sophisticated use of artificial intelligence to maximize support for the user's mental health and overall well-being.

In addition to the aforementioned operations, the system further includes a step of generating a customized emotion management report for the user. This report provides a comprehensive overview of the emotional state of the user, the recommended management strategies, and any progress or changes that may have occurred over time. The report can include visual representations such as charts or graphs to illustrate trends in the user's emotional states and the effectiveness of the recommended strategies. The report is personalized, basing its content on the user's specific emotional input, the successive interactions with the system, and the analytics tracked by the analytics module.

Overall, the disclosed system and method signify a sophisticated combination of AI technologies, personalized data analytics, and user interaction to provide real-time, tailored emotional management recommendations in a manner that is sensitive to the individual needs of the user.

Emotional state inputs are gathered from users (S100) through a user-friendly mobile application interface. The mobile application is designed to be engaging and easily navigable, ensuring users can comfortably convey their current emotional states, including feelings of anxiety or stress. The emotional state input is received in multiple formats, such as text, voice, or even physiological signals, should compatible hardware be available.

The system also observes user progress in emotional management over time. Tracking longitudinal changes allows the AI to gain insights into the efficacy of the recommendations and their impact on the user's emotional health. It can identify patterns, such as which strategies yield the best results, and adapt future recommendations to lean into these successful tactics. The feedback loop created by monitoring user progress over time ensures that the AI psychologist module continuously evolves. As a result, the system refines its understanding of the user's emotional wellbeing journey, becoming more precise and beneficial with each interaction. The result is a seamlessly integrated and intelligently adaptive aid to emotional self-management, which sets new standards in personalized care. Users benefit from an AI companion that understands the intricacies of their emotional landscape and provides substantiated guidance that can evolve alongside their personal growth and changing needs.

By tracking these emotional profiles over time, the system can identify patterns in the user's emotional responses, such as specific triggers that may lead to heightened states of anxiety or moments of particular joy. The significance of such patterns can be correlated with user-specific information such as historical data, calendar events, reported stressors, or environmental factors that may influence emotional states.

The AI psychologist module generates these exercises by assessing a variety of factors such as the user's past responses to different kinds of interventions, the time of day, contextual factors derived from the user's environment, and any other information deemed relevant from the knowledge base. In doing so, the system ensures that each exercise is not only theoretically effective but also practically applicable within the specific situational context of the user's life.

Moreover, these self-consciousness and self-enhancement exercises are seamlessly integrated into the mobile application interface, ensuring that users have easy access to their personalized recommendations and exercises without having to navigate away from the primary application environment. This integration helps maintain user engagement by offering a single, coherent source for emotional well-being. In essence, the inclusion of self-consciousness and self-enhancement exercises broadens the scope of the AI psychologist module's functionality, thereby providing a more holistic approach to emotional state management that goes beyond reactive recommendations to proactive personal development.

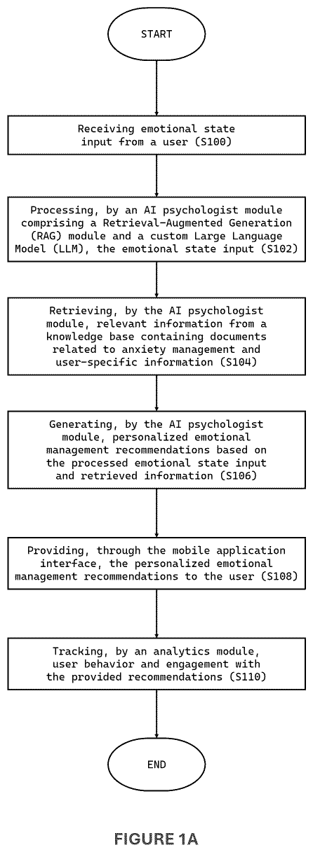

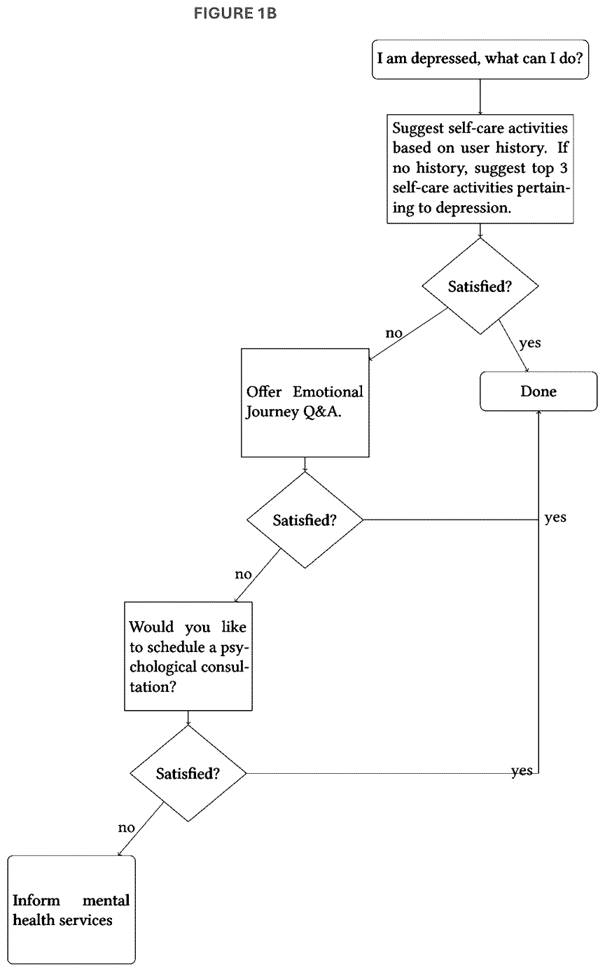

FIG. 1B shows an exemplary operation of the system of FIG. 1A. In this example, the user inputs that he/she is depressed and request help. The system suggests self-care activities based on the user history. If there is no patient history, the system suggests top three self-care activities pertaining to depression. The system checks if the user is satisfied and if so exits. If not, the system offers an emotional journey question and answer (Q&A) session and checks for user satisfaction. If the user is still not satisfied, the system checks whether the user is satisfied. If not, the system refers the user to a psychological consultation and again check for satisfaction. If the user is still unhappy, the system informs mental health services to get assistance for the user.

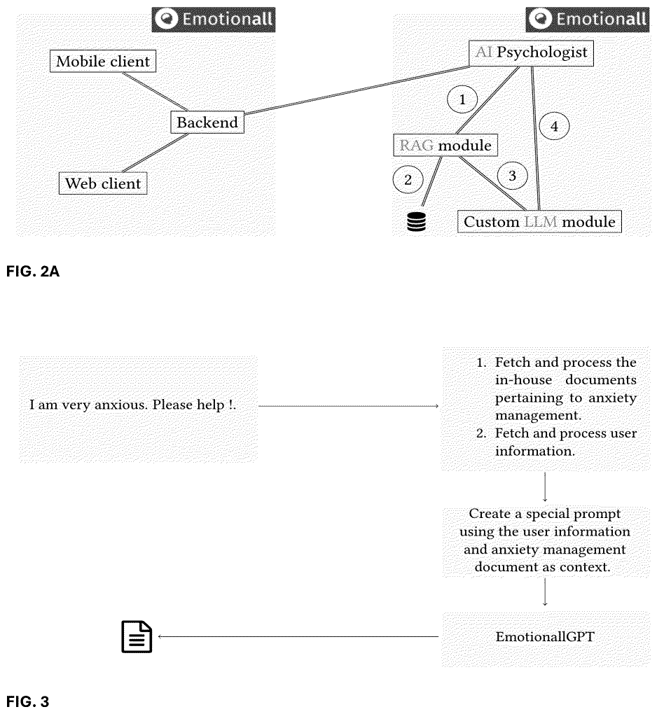

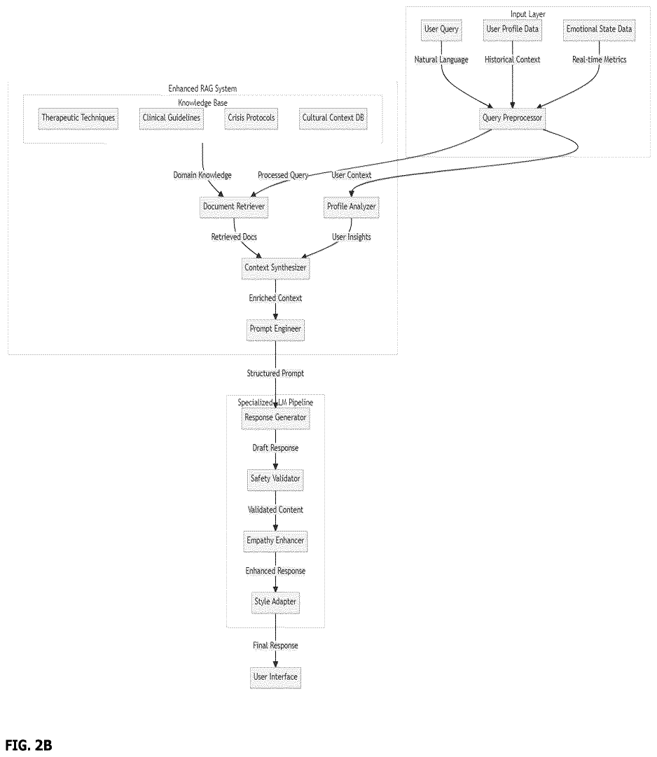

FIG. 2A shows an AI Psychologist module, which integrates Retrieval-Augmented Generation (RAG) with a database and a custom Large Language Model (LLM). The AI Psychologist system enables the app to analyze user emotions, retrieve contextually relevant insights, and deliver tailored recommendations. With features including emotional surveys, progress tracking, and a dynamic AI chatbot, the system provides a comprehensive toolkit for users to achieve emotional balance and long-term mental health improvement. When a user submits a query, the backend retrieves relevant information from its in-house knowledge base. This includes processing documents and user-specific details to create a structured input for the custom LLM. The LLM then generates a tailored response, offering users clear and actionable guidance.

The AI Psychologist module delivers personalized emotional support, insights, and recommendations. It uniquely integrates a Retrieval-Augmented Generation (RAG) framework with a customized Large Language Model (LLM), enabling it to process user queries with precision and empathy. Unlike generic AI systems, the module combines user-specific data, curated domain knowledge, and advanced NLP techniques to generate context-aware responses. Its primary objective is to help users manage their emotional states, improve mental well-being, and foster positive behavioral changes.

FIG. 2B shows one implementation of FIG. 2A. The architectural diagram illustrates a AI-driven emotional management system that processes user interactions through multiple interconnected components. The system begins with an Input Layer that accepts three primary data streams: User Query, User Profile Data, and Emotional State Data. These inputs are processed through a Query Preprocessor that handles Natural Language, Historical Context, and Real-time Metrics.

A RAG System includes a Knowledge Base with four key components: Therapeutic Techniques, Clinical Guidelines, Crisis Protocols, and Cultural Context database. The Document Retriever works in parallel with a Profile Analyzer to process Domain Knowledge, Processed Query, and User Context, generating Retrieved Docs and User Insights respectively.

The Context Synthesizer merges these outputs to create an Enriched Context, which is then passed to an LLM Prompt Engineering module that generates a Structured Prompt. This structured input feeds into a Specialized LLM Pipeline, which consists of multiple processing stages. First, a Response Generator creates a Draft Response, which then passes through a Safety Validator to ensure content appropriateness. The validated content moves through an Empathy Enhancer to add emotional intelligence, followed by a Style Adapter that refines the presentation. The final stage of the pipeline delivers the Final Response through a User Interface, completing the workflow. This architecture enables real-time processing of emotional states while maintaining privacy and ensuring appropriate emotional responses through multiple validation and enhancement stages. The system's modular design allows for continuous improvement and adaptation of responses based on user interaction and feedback, making it particularly effective for providing personalized psychological support.

The Input Layer has three parallel data streams: User Query (natural language input), User Profile Data (historical context), and Emotional State Data (real-time metrics). These inputs feed into a Query Preprocessor that handles natural language processing, historical context analysis, and real-time metric processing.

The Enhanced RAG System contains a Knowledge Base with four components: Therapeutic Techniques, Clinical Guidelines, Crisis Protocols and Cultural Context DB. The knowledge base connects to two parallel processing components:
Document Retriever: Processes Domain Knowledge and Processed Query
Profile Analyzer: Processes User Context
The outputs from both processors flow into the Context Synthesizer which combines Retrieved Docs and User Insights and into the Prompt Engineer that transforms Enriched Context into Structured Prompt

The Response Generation Pipeline includes the Specialized LLM Pipeline which processes the structured prompt through sequential stages: the Response Generator creates initial Draft Response, the Safety Validator ensures content appropriateness, the Empathy Enhancer adds emotional intelligence, and the Style Adapter refines presentation. The final stage delivers the processed response through a User Interface, completing the workflow while maintaining continuous feedback loops for system improvement. The system's architecture enables real-time processing while maintaining privacy and ensuring appropriate emotional responses through multiple validation and enhancement stages.

In one exemplary operation, based on the provided input, the AI psychologist fetches the required document from the curated knowledge base. Along with the document, relevant user information are also acquired. Once the data has been acquired, it further needs to be cleaned and processed in a format that can be understood by the LLM. There are two approaches to data preparation in this case:

(a) Function calling: Function calling enables a LLM to efficiently integrate with external systems. For example, consider the task of searching the internet for a relevant document. Based on the input provided, the LLM can be used to generate a request object which can then be automatically used to perform the online search. Once the document has been acquired it can be chunked and processed subsequently to generate the appropriate prompt.

(b) Embedding search on a vector database: The relevant documents can be acquired, processed and stored in a vector database through a one-time operation. When the user provides the input, it is used to perform an embedding search in the vector database to fetch relevant documents. Finally, the resultant set of documents can be used to generate the appropriate prompt.

After data preparation, prompt generation can be done. Once the required data has been processed, it needs to be added to the prompt for the LLM in a particular manner. Te generated prompt is passed to a custom LLM in a particular format for inference. The LLM processes the specially engineered prompt and generates the output in text format.

### 2.3.2 Input Data

The system processes three distinct types of input data to ensure contextually relevant outputs:

### 1. User Queries

These are natural language inputs provided by users, often describing emotional states or seeking advice.
Example: A user may ask, “I feel anxious and overwhelmed. What should I do?”

### 2. User-Specific Information

Includes emotional history, survey responses, activity feedback, and other contextual data.
For example, a user with a history of high anxiety levels may have prior recommendations or activities logged in the system.

### 3. Knowledge Base

repository of in-house documents focusing on psychological techniques, anxiety management, and emotional well-being.
It includes structured content such as therapeutic guidelines, stress management exercises, and mindfulness practices.

The integration of these data sources ensures the module can provide responses tailored to individual needs while maintaining general applicability.

The Retrieval-Augmented Generation (RAG) module is responsible for retrieving relevant data to enrich the context of user queries. It functions as follows:

### 1. Document Retrieval

The module uses semantic search and vector embeddings to identify and fetch documents from the knowledge base that align with the user's query.
For instance, a query about “anxiety” triggers the retrieval of anxiety management guides, cognitive behavioral therapy (CBT) exercises, and breathing techniques.

### 2. User Context Retrieval

Retrieves user-specific emotional history, recent survey results, and activity logs.
Example: If a user frequently reports high stress levels, the system prioritizes stress-relief techniques in its context retrieval.

### 3. Context Creation

Combines the retrieved documents with user-specific information to create a comprehensive context for processing.
This structured input enables the LLM to generate responses that are both accurate and personalized.
   The Large Language Model (LLM) processes the structured input generated by the RAG module to provide actionable and empathetic responses. Its operation can be broken down into three stages:

### 1. Prompt Creation

Dynamically constructs a detailed prompt that integrates:
   The user query.
   Context from the retrieved documents and user history.
   Task-specific instructions (e.g., “Provide actionable advice on managing anxiety.”).
Example Prompt “User query: ‘I feel anxious.’
Retrieved context: ‘Anxiety management document, breathing exercises.’ Instruction: ‘Generate a supportive, empathetic response.’”

### 2. Response Generation

Processes the prompt to generate a detailed, empathetic, and actionable response.
Example Response: “It's okay to feel anxious. Here's a technique that may help: Try deep breathing exercises—inhale for 4 seconds, hold for 7 seconds, and exhale for 8 seconds. This can help reduce immediate stress. Would you like additional tips?”

### 3. Output Refinement

Applies post-processing steps to ensure the response is:
   Therapeutically aligned.
   Grammatically correct and easy to understand.
   Free from biases or inaccuracies.
      The custom LLM is developed through a multi-phase training process, tailored specifically for emotional intelligence and therapeutic applications:

### 1. Pre-Training:

The base model is trained on a large corpus of general text, including books, articles, and conversations, to develop a foundational understanding of language.

### 2. Fine-Tuning:

Fine-tuned on datasets specific to emotional well-being, such as:
   Psychological literature and clinical guidelines.
   Therapy session transcripts.
   Emotional support conversations and self-care recommendations.
      3. Reinforcement Learning with Human Feedback (RLHF):
Human evaluators provide feedback on model outputs to optimize for:
   Empathy and supportiveness.
   Relevance to the user's context.
   Therapeutic soundness.

### 4. Continuous Learning:

User interactions are anonymized and analyzed to improve future responses.
Feedback loops ensure the system evolves to better address user needs.

```
function AIPsychologistModule(userQuery, userContext):
  // Step 1: RAG Operation
  relevantDocuments = retrieveRelevantDocuments(userQuery)
  userSpecificData = retrieveUserContext(userContext)
  // Step 2: Context Creation
  enrichedContext = combineContext(relevantDocuments,
  userSpecificData)
  // Step 3: LLM Operation
  prompt = createPrompt(userQuery, enrichedContext)
  response = generateLLMResponse(prompt)
  refinedResponse = refineOutput(response)
  return refinedResponse
function retrieveRelevantDocuments(query):
  // Use semantic search and vector embeddings to find relevant
  documents
  return relevantDocs
function retrieveUserContext(userContext):
  // Fetch user-specific emotional history, survey results, and activity
  logs
  return userData
function combineContext(documents, userData):
  // Merge retrieved documents with user-specific information
  return combinedContext
function createPrompt(query, context):
  // Construct a detailed prompt integrating query, context, and
  instructions
  return formattedPrompt
function generateLLMResponse(prompt):
  // Process the prompt using the fine-tuned LLM
  return rawResponse
function refineOutput(response):
  // Apply post-processing for therapeutic alignment and bias mitigation
  return refinedResponse
// Training process (performed separately)
function trainAIPsychologist( ):
  baseModel = preTrainOnGeneralCorpus( )
  fineTunedModel = fineTuneOnEmotionalWellbeingData(baseModel)
  optimizedModel = applyReinforcementLearning(fineTunedModel)
  return optimizedModel
// Continuous learning
function updateModel(userFeedback):
  anonymizedData = anonymizeUserData(userFeedback)
  improvedModel = incorporateFeedback(currentModel,
  anonymizedData)
  return improvedModel
```

The AI Psychologist module sets itself apart from other RAG/LLM systems through the following features:

### 1. Personalization:

Combines user-specific emotional data and contextual information to generate tailored responses.
Example: For a user with prior history of anxiety, the system prioritizes relevant coping strategies.

### 2. Domain-Specific Fine-Tuning:

Fine-tuned exclusively on datasets relevant to emotional intelligence and mental health, making it uniquely suited for therapeutic applications.

### 3. Bias Mitigation:

Incorporates techniques like adversarial debiasing and counterfactual fairness to minimize biases in generated responses.

### 4. Privacy and Security:

Sensitive user data is anonymized and encrypted to ensure compliance with privacy regulations.
Only necessary data is processed to maintain user confidentiality.

### 5. Therapeutic Alignment:

All generated responses adhere to clinical guidelines and best practices in mental health support.

| Feature | Emotionall's RAG/LLM system | Generic RAG/LLM Systems |
|---|---|---|
| Domain-Specific | Fine-tuned exclusively on psychological | Typically pre-trained on |
| Fine-Tuning | datasets, therapy session transcripts, and | large, generic corpora and |
| | emotional support literature. | lacks specific adaptation for |
| | | mental health. |
| Personalization | Integrates user-specific emotional | Limited or no personalization; |
| | history, survey responses, and context | relies solely on generic |
| | for tailored outputs. | prompts without user-specific |
| | | data. |
| Therapeutic | Responses adhere to clinical guidelines | Responses may lack |
| Alignment | and best practices in mental health and | therapeutic validation and |
| | emotional well-being. | alignment with clinical |
| | | standards. |
| RAG Optimization | Combines user context with domain- | Often limited to static |
| | specific document retrieval to enrich | knowledge base retrieval with |
| | LLM prompts. | less emphasis on user-specific |
| | | contextualization. |
| Bias Mitigation | Employs adversarial debiasing and | Generic models may |
| | counterfactual fairness techniques to | perpetuate biases present in |
| | reduce biases in responses. | training data with limited |
| | | debiasing capabilities. |
| Privacy and | Anonymizes sensitive user data and uses | May lack robust privacy |
| Security | encryption to ensure compliance with | measures, especially when |
| | GDPR and other standards. | integrating third-party APIs. |
| Continuous | Incorporates user feedback loops for | Often relies on static updates |
| Learning | iterative improvement in both RAG and | without user-driven |
| | LLM components. | refinement. |

FIG. 3 shows an example use-case. In this example, when a user submits a query “I am very anxious. Please help!”, the system identifies and retrieves relevant documents related to anxiety management from a knowledge database. The system combines this information with the user's context to create a detailed input for the LLM. The LLM processes this input and provides a personalized response to address the user's emotional needs. By combining AI technologies with a focus on user experience, the system offers a unique solution for emotional well-being with personalized support and meaningful insights, helping users manage their emotions and improve their mental health effectively.

In certain embodiments, the artificial intelligence (AI) psychologist module further includes a motivational enhancement component, leveraging its advanced processing and insightful analysis from both the RAG module and the custom LLM to not only provide emotional management recommendations but also to recommend activities specifically designed to enhance the user's motivation and social skills. Upon receiving the emotional state input from the user, indicative of feelings such as anxiety or demotivation, the system dynamically integrates this information to tailor activities that are conducive to uplifting the user's spirits and improving their interpersonal interactions.

The recommendation of activities by the AI psychologist module goes beyond generic advice, as it considers specific characteristics of the user, such as their past behavior, engagement patterns, preference for solitary or group tasks, and their historical response to previous recommendations. These factors are critical in personalizing the experience and ensuring relevance, which in turn, supports the efficacy of the intervention. For example, users who thrive in social settings may receive recommendations for group activities that encourage socializing, while users who prefer solitary activities may be suggested tasks that can be performed alone but are still effective in enhancing motivation and social capabilities indirectly.

This dynamic system ensures that the users are not merely given a static set of recommendations but are instead provided with an evolving, adaptive, and interactive tool for managing their emotional states. It recognizes the nuanced nature of emotional experiences and the value of motivation and social interaction in enhancing mental well-being. Thus, the system not only aids with immediate emotional regulation but also fosters long-term resilience through skill enhancement and motivational support.

These personalized recommendations are then delivered to the user through the application interface, a step depicted by S108. The design ensures that the recommendations are easily accessible and actionable for the user, enhancing the overall experience and the likelihood of positive emotional management by the user.

By analyzing user behavior and engagement patterns, the system can refine its future recommendations, making them increasingly personalized and effective. This continuous improvement cycle is designed to help users not only manage negative emotions but also accentuate positive emotions.

Through its targeted and personalized recommendations, one implementation aims to foster an increase in positive emotions and a reduction in negative emotions. By doing so, the system may contribute to the prevention of mental illnesses that are typically associated with prolonged negative emotional experiences. By mitigating these risks before they can manifest as more severe issues, one implementation outlines a proactive approach to emotional well-being, setting a new standard in the intersection of technology and mental health wellness.

Guiding the user to live in accordance with their personal values and purpose. To achieve this, the AI psychologist module employs a sophisticated algorithm that takes into account not only the emotional state and behavioral engagement data but also integrates user input regarding their personal values and life objectives. This data may be gathered through initial setup questionnaires, ongoing user input, and inferred from user interactions with the system.

Moreover, the system leverages the analytics module to monitor how the user engages with the value- and purpose-aligned guidance. By tracking this engagement (S110), the system can further personalize future recommendations in order to continuously refine the support it offers. The system may also incorporate feedback mechanisms allowing the user to reflect on the extent to which the recommendations have helped to promote actions and decisions aligned with their personal values and life goals.

In this manner, the system goes beyond traditional emotional support mechanisms and acts as a comprehensive tool for personal development and emotional well-being. By taking into account the multidimensional aspects of a user's mental health, including alignment with personal values and life purpose, the disclosed one implementation provides an innovative approach to integrative emotional management.

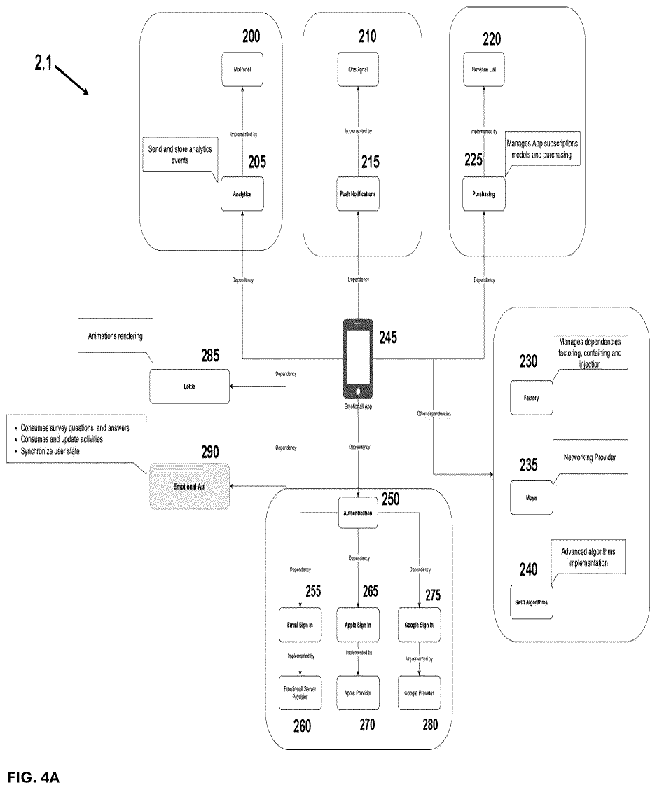

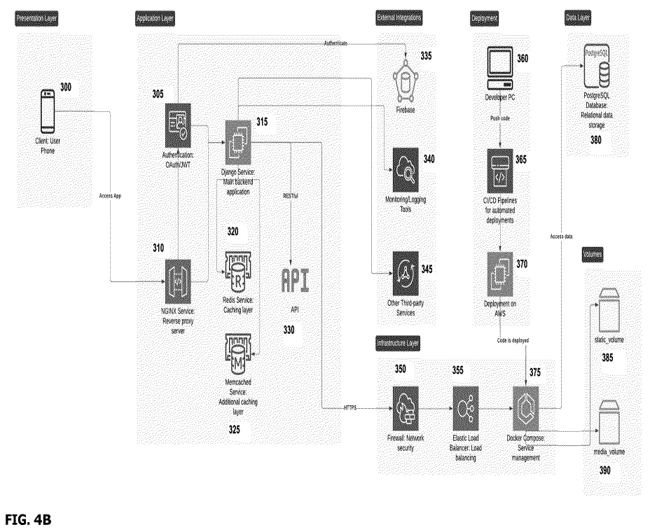

FIG. 4A shows a front-end architecture diagram of an exemplary emotional mobile app 245, the core mobile interface through which users interact with various features and services. The app relies on multiple modules, each responsible for different aspects of functionality. Starting with Analytics (200-205), implemented by MixPanel—200, this module is used for tracking user behavior and engagement by sending and storing analytics events. This data collection helps improve user experience based on interaction insights. Push Notifications (210-215), powered by OneSignal—210, enables the app to send timely updates and reminders directly to users, promoting engagement through real-time alerts.

The Purchasing (220-225) module, managed by Revenue Cat—220, handles all in-app subscriptions and purchases. It facilitates a seamless subscription model, allowing users to upgrade their accounts and manage their subscriptions within the app. Moving on to Dependency Management (230-240), this section includes essential components like Factory—230, which manages dependency injection and containment, ensuring that each module has access to the resources it needs. Networking Provider—235 handles all API requests and external communications, while Swift Algorithms—240 provides a set of advanced algorithms to support complex computations and processes within the app.

Authentication (250-280) is a critical module for user access and account management, allowing users to sign in through various methods. This includes Email Sign-in 255, facilitated by the Emotional Server Provider—260, Apple Sign-in (265) implemented by the Apple Provider (270), and Google Sign-in (275) managed by the Google Provider (280). These options provide flexibility and convenience for users to securely access their accounts.

Lottie (285) is responsible for rendering animations within the app, enhancing the visual appeal and making the user interface more dynamic. Finally, the Emotional API (290) serves as the backend communication layer that synchronizes user data, such as survey responses, activities, and emotional states. It allows the app to fetch and update user-specific information, ensuring a personalized experience.

Together, these modules and dependencies form a modular and scalable architecture, where each component operates independently but connects seamlessly with the Emotional App. This structure enables efficient integration with third-party services, modular development, and a streamlined user experience, allowing for robust functionality and easy maintenance of the app.

As shown in FIG. 4B, an exemplary backend architecture diagram revolves around the system's core backend services and infrastructure, ensuring scalability, security, and efficiency. Starting with the Presentation Layer Step 300, represented by Client: User Phone Step 300, the mobile interface allows users to interact with the application, accessing features and services seamlessly. The Authentication Module-Step 305, powered by OAuth/JWT-Step 305, ensures secure user login and API authorization, allowing only verified users to access system resources. The Reverse Proxy Layer-Step 310, managed by NGINX Service-Step 310, acts as the entry point for incoming requests, distributing them across backend services, enhancing performance, and securing communications. The Backend Core Step (315-325) includes the Django Service: Main Backend Application Step—315, which handles business logic, processes API requests, and communicates with the database. Supporting this is the Redis Service: Caching Layer-Step 320 and Memcached Service: Additional Caching Layer—Step 325, which optimize performance by caching frequently accessed data. The API Layer—Step 330 provides RESTful endpoints for external communication, enabling integrations and smooth data exchange. The External Integrations Step (335-345) include Firebase Step—335 for notifications and real-time database functionalities, Monitoring/Logging Tools—Step 340 to track performance and errors, and Other Third-party Services—Step 345 that extend the application's capabilities. The Deployment Pipeline Step (360-375) begins with the Developer PC—Step 360, where code is written and tested before being pushed to the CI/CD Pipelines for Automated Deployments (365). The final deployment occurs on AWS Step—370, ensuring a scalable and reliable infrastructure. Docker Compose Step—375 manages containerized services, ensuring consistent environments across deployments. The Infrastructure Layer Step (350-355) ensures security and scalability with a Firewall: Network Security Step—350 to filter malicious traffic and an Elastic Load Balancer—Step 355 to distribute requests evenly across servers. Finally, the Data Layer Step—(380-390) consists of the PostgreSQL Database: Relational Data Storage—Step 380 for structured data and the Volume Management System Step (385-390), which includes static_volume Step—385 for static files and media_volume—Step 390 for user-uploaded content.

FIG. 5 shows an exemplary emotional survey process. The emotional survey Flow 432a begins with the user submitting survey data through a POST request, initiating the survey processing sequence at Step 775. Upon receiving the data, the system first validates the submitted information at Step 780, ensuring that the responses conform to the required formats and contain all necessary fields. Following validation, the system checks if the data is valid at Step 785. If the data fails this check, an error response is returned to the user at Step 840, informing them of validation issues that must be resolved before resubmitting. However, if the data is valid, the flow proceeds to check if the user has previously submitted responses for this survey at Step 790.

If the system detects an existing submission from the user, it immediately responds with an error message stating “Already submitted” at Step 795, thereby preventing duplicate entries. If no prior submission is found, the system creates an EmotionalSurveyResponse entry for each question in the survey at Step 800, saving the user's responses to ensure accurate records per FIG. 5 and Table 1—surveys_emotionalsurveyresponse. Next, the system verifies the integrity of these responses at Step 805, identifying any duplicate answers for a single question. Should an integrity error be detected, the system sends an error response to the user with the message “Multiple responses on one question” at Step 810, indicating that only one response per question is allowed.

With no integrity issues, the flow advances to check whether an emotional state record in Step 421—accounts_useremotionalstate in FIG. 5, already exists for the user at Step 815. If an emotional state record is not found at Step 820, a new emotional state entry is created for the user at Step 825. This step ensures that each user has an emotional profile that can be updated and referenced for personalised recommendations. The system then updates the user's recommended activities based on the latest survey responses and emotional state at Step 830, tailoring suggestions to support the user's emotional well-being. Finally, upon completing these steps, the system responds with a success message at Step 835, confirming that the survey has been processed and recommendations have been updated accordingly. The flow concludes after sending either a success or error response, marking the end of the process.

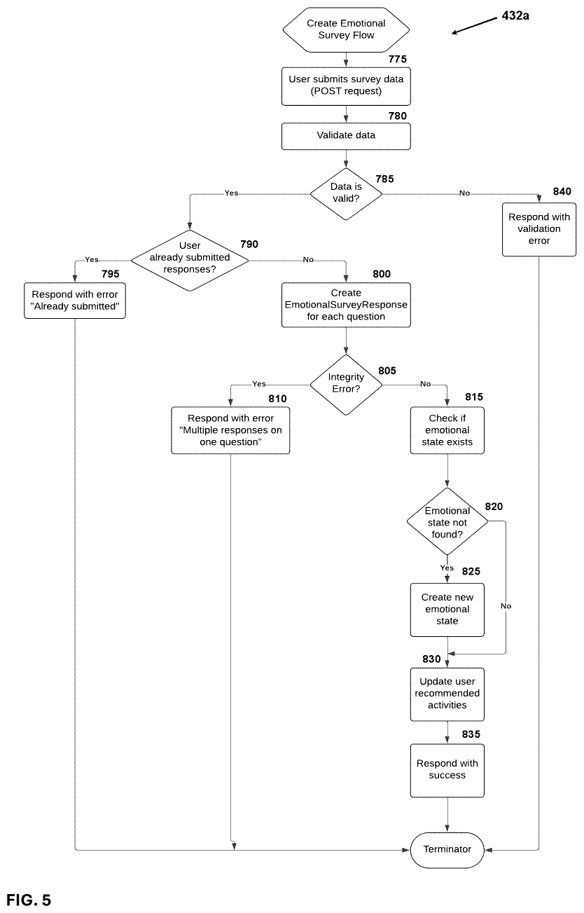

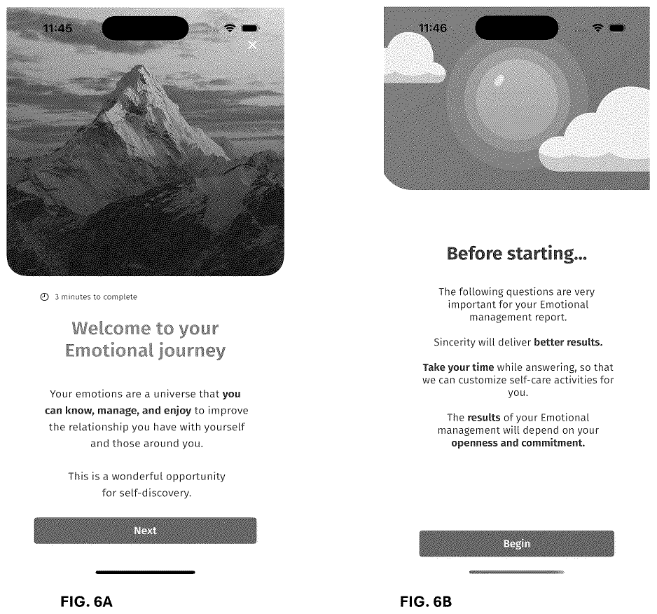

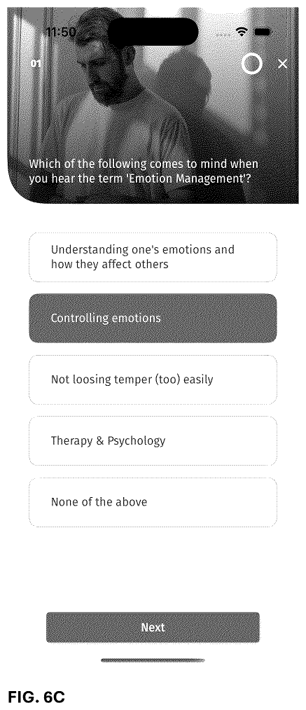

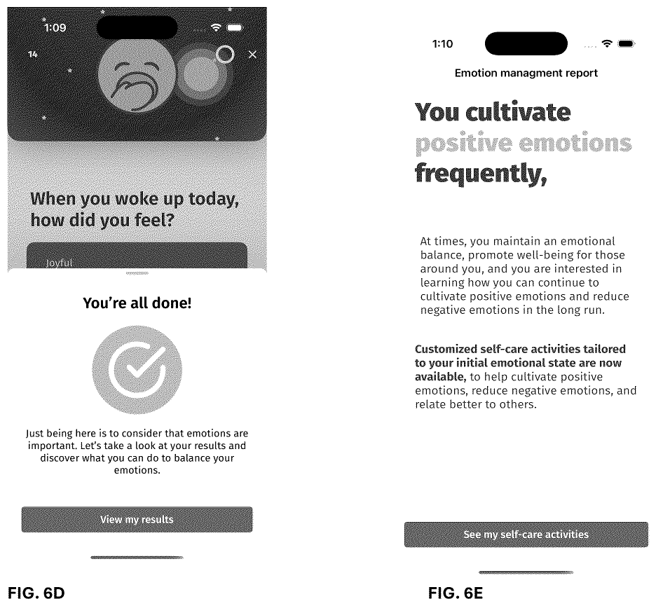

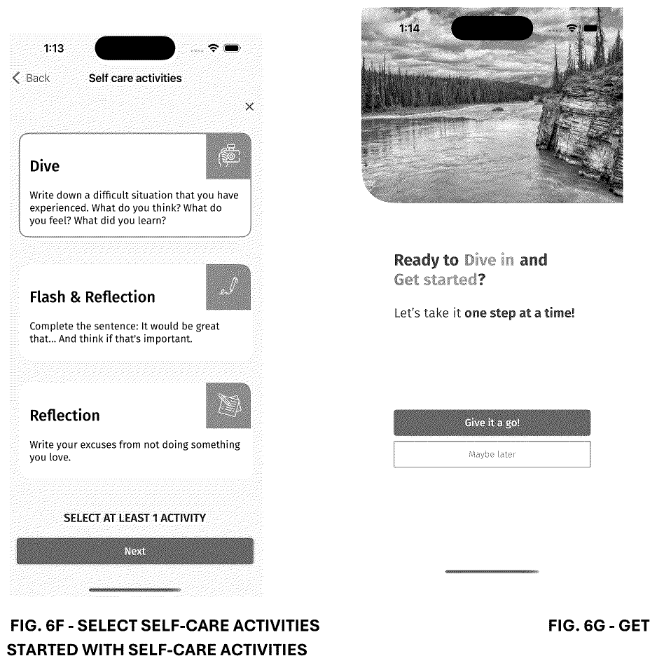

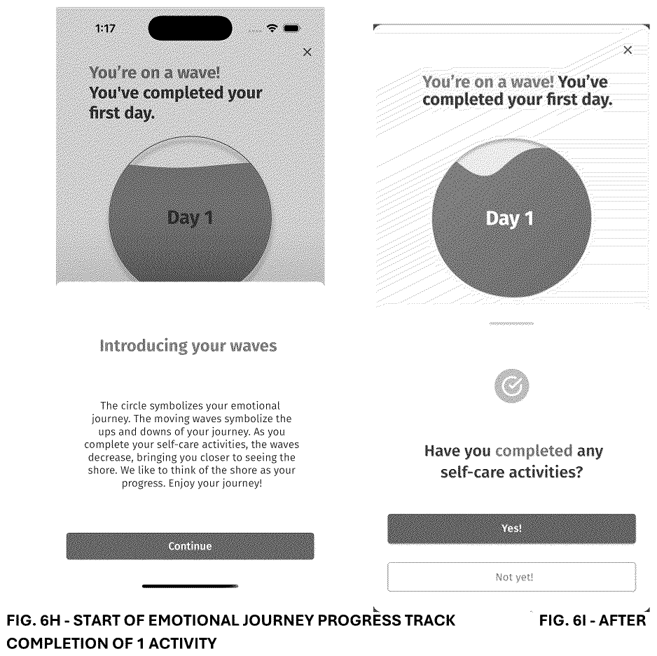

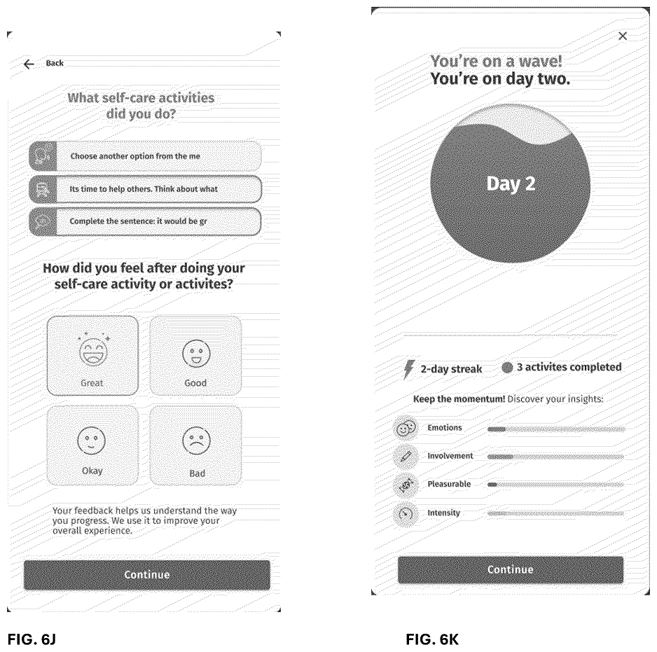

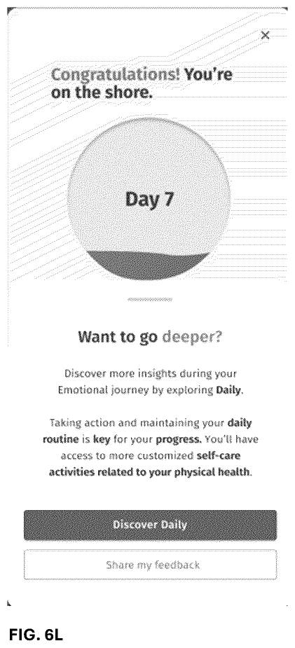

FIG. 6A-6L show exemplary user interface screenshots for the preferred embodiment. When the user begins the Emotional Survey Flow, FIG. 6A and FIG. 6B will be displayed as Intro pages on emotion management. FIG. 6C shows an exemplary layout of questions and options where during Step 800, this UI will be shown with Questions and Options related to Emotional Survey Flow.

Below in Table-1, the user will be asked to answer these many questions with the options provided:

TABLE 1
Emotional Survey Flow Questions with Options

| Q. | Questions | Options | |
|---|---|---|---|
| 1 | Which of the following comes to mind | a) | Understanding one's emotions and |
| | when you hear the term ‘Emotion | | how they affect others |
| | Management’? | b) | Controlling emotions |
| | | c) | Not losing temper (too) easily |
| | | d) | Therapy, psychology |
| | | e) | None of the above |
| 2 | Did you recently experience one of the | a) | Having a discussion with my partner |
| | following situations which required you | b) | Interacting with a coworker |
| | to manage your emotions? | c) | Speaking in public |
| | | d) | Expecting something bad will happen |
| | | e) | None of the above |
| 3 | How would you react if someone, out of | a) | Aggressive |
| | the blue, crashed into your car? | b) | Arguing heavily |
| | | c) | Discussing |
| | | d) | Negotiating |
| | | e) | Calm |
| 4 | Did you experience one of the following | a) | Sadness |
| | (negative) emotions today? | b) | Frustration |
| | | c) | Anger |
| | | d) | Fear |
| | | e) | None of the above |
| 5 | Did you experience one of the following | a) | Love |
| | (positive) emotions today? | b) | Gratitude |
| | | c) | Joy |
| | | d) | Pride |
| | | e) | None of the above |
| 6 | Do you consider that you can | a) | Yes |
| | successfully manage your emotions? | b) | No |
| 7 | Did you express your gratitude to | a) | Yes |
| | someone today? | b) | No |
| 8 | Do you prevent others from imposing | a) | Always |
| | their wishes over your own principles | b) | Oftentimes |
| | and values? | c) | Sometimes |
| | | d) | Almost never |
| | | e) | Never |
| 9 | Did you check in with a loved one today | a) | Yes |
| | just to see how they are doing? | b) | No |
| 10 | Did something happen today that had a | a) | Yes |
| | positive impact on you? | b) | No |
| 11 | Did you show someone today that you | a) | Yes |
| | care about something? | b) | No |
| 12 | Did you share your feelings with | a) | Yes |
| | someone today? | b) | No |
| 13 | Did you make someone smile today? | a) | Yes |
| | | b) | No |
| 14 | When you woke up today, how did you | a) | Joyful |
| | feel? | b) | Calmed |
| | | c) | Sad |
| | | d) | Down |
| | | e) | Angry |

After completion of all questions of Emotional Survey, the success page will be shown as FIG. 6D and this process happens in Step 835. After clicking on the button “View my results in FIG. 6D, FIG. 6E will be shown which is the Emotion Management Report.

After completion of the Emotional Survey, the system provides 3 options for self-care activities: Dive, Flash & Reflection and Reflection, which is shown in FIG. 6D. Once the user selects at least one activity, FIG. 6E will be displayed to start the activity.

The Emotional Journey Progress Tracking process is detailed next. After completion of Emotional SurveyFlow, the user is directed to an Emotional Journey Progress Tracking UI in FIG. 6H over a predetermined period such as 7 days in one implementation. After completion of one self-care activity in Day 1, FIG. 6I is shown. In FIG. 6I there are options whether the user has completed the activity or not. If completed, user selects ‘Yes!’, which will proceed to FIG. 6J.

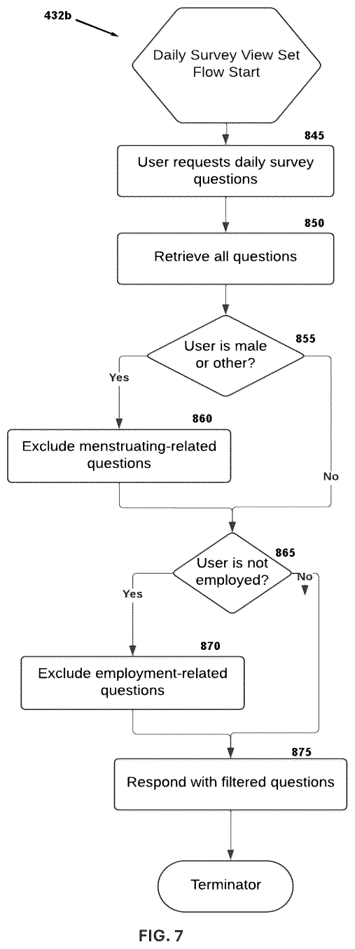

FIG. 6J shows a survey post self-care activity UI which collects feedback for the overall experience of the user. After continuing, FIG. 6K will appear, which follows the same-doing at least 1 self-care activity out of 3 and maintaining the day streak. This process is completed for each day of the program. After completion of 7-Day self-care activity FIG. 6L will be displayed, which shows 2 options: a) Discover Daily and b) Share my Feedback. If you select Option a, then a Daily Survey Flow process detailed in FIG. 7 will begin.

The Daily Survey View Set Flow (FIG. 7) begins when the user initiates a request for daily survey questions, marking the flow's start at Node 432b. In Step 845, the user sends a request to retrieve the set of daily survey questions from Table 450. Upon receiving this request, the system retrieves all available questions at Step 850, preparing them for filtering based on the user's characteristics. The next step, Step 855, assesses whether the user is identified as male or has selected “other” for gender. If the answer is “Yes,” the system excludes menstruation-related questions at Step 860, removing questions that may not be relevant to this user. If the answer is “No,” indicating the user may require menstruation-related content, the flow continues without filtering these questions.

Following the gender check, the flow proceeds to Step 865, where the system examines the user's employment status. If the user is not employed, the flow moves to Step 870, excluding employment-related questions that may be irrelevant to their daily experience. If the user is employed, the system retains employment-related questions, ensuring that relevant content remains in the survey set. Finally, after completing the necessary filtering steps, the system responds with the curated set of questions at Step 875, presenting the user with only the most pertinent questions based on their profile. The flow concludes after delivering the filtered questions, marking the end of the Daily Survey View Set Flow.

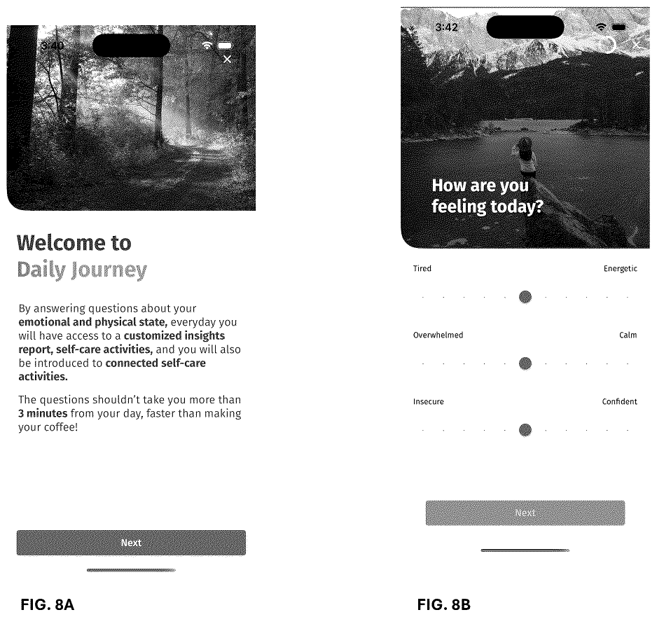

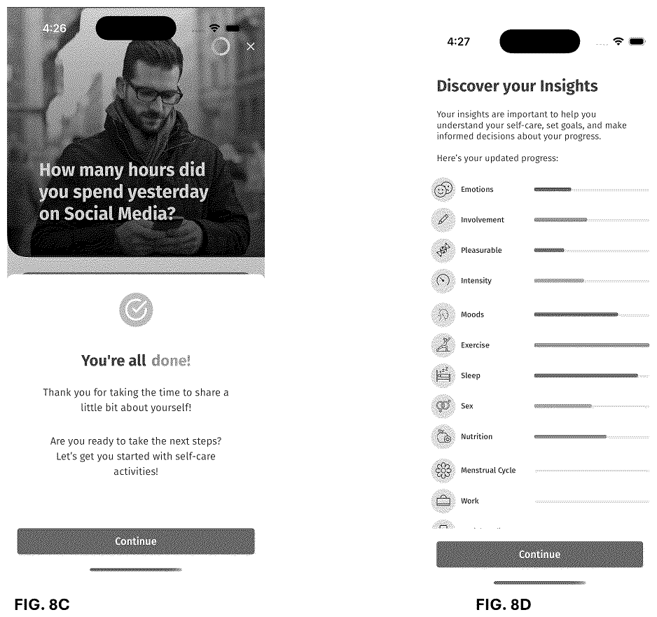

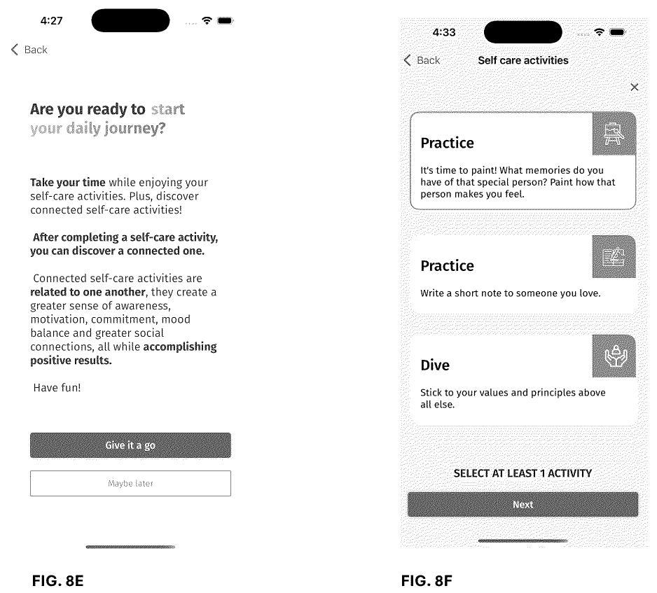

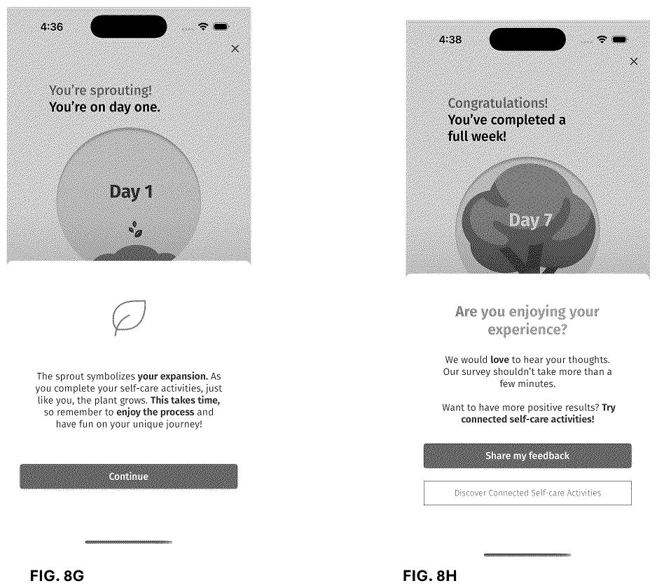

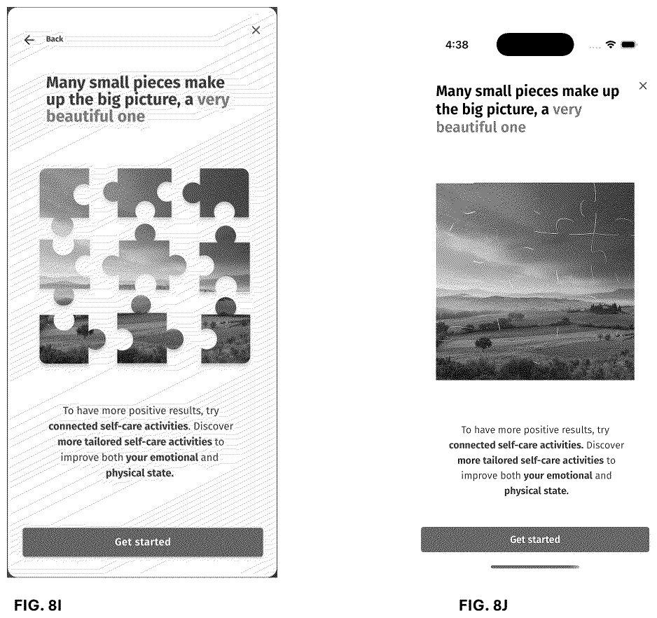

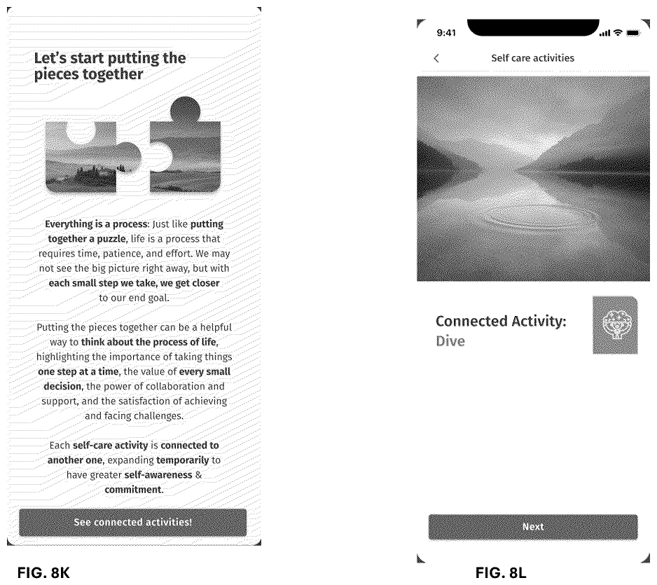

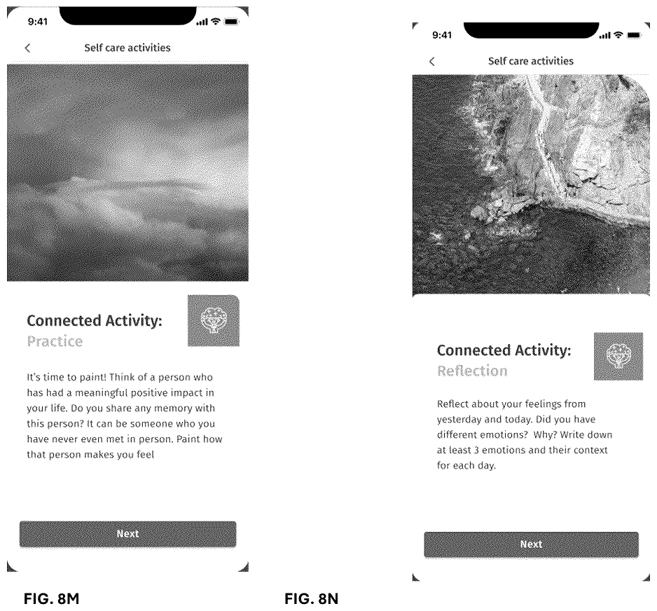

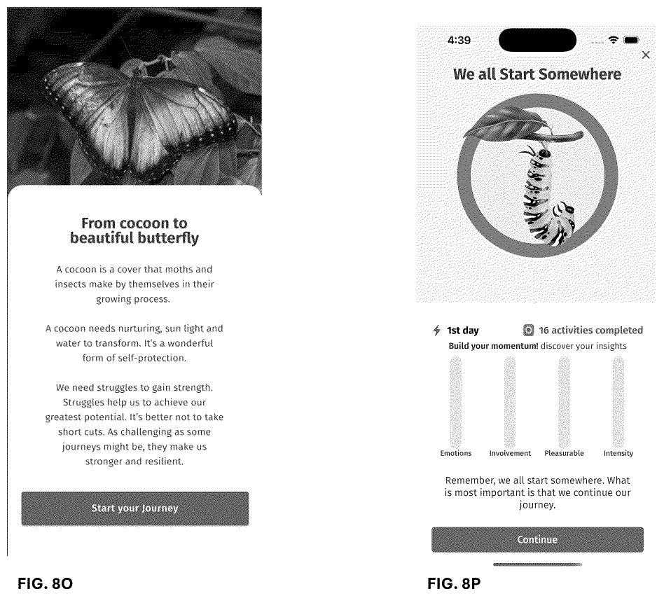

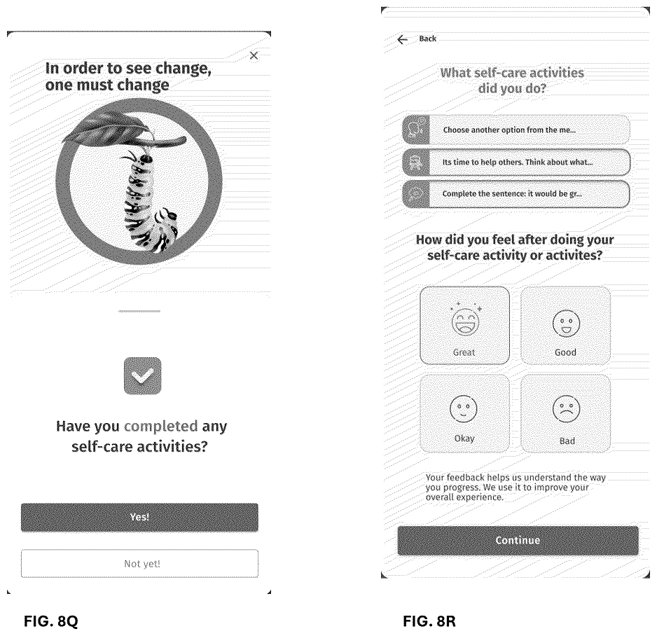

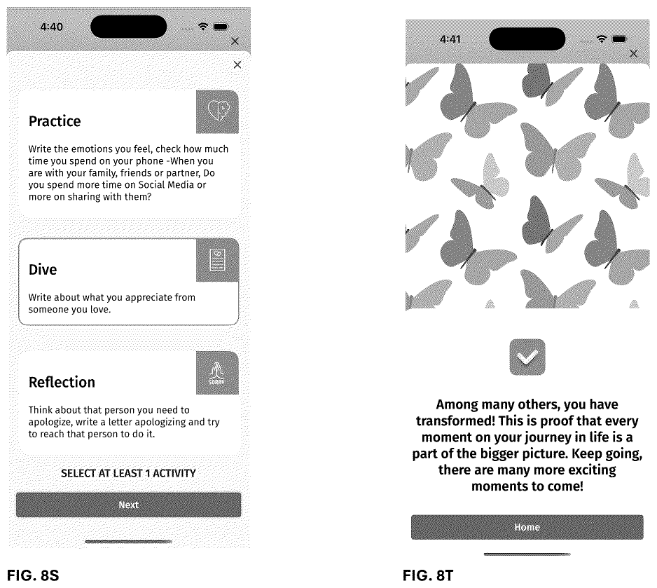

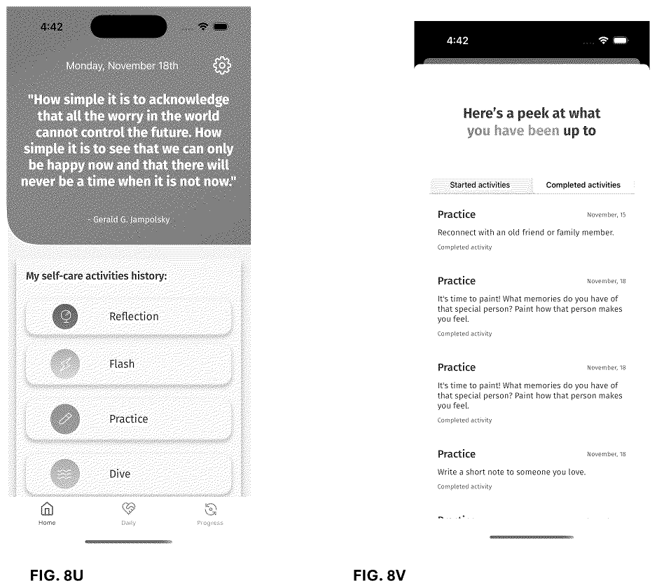

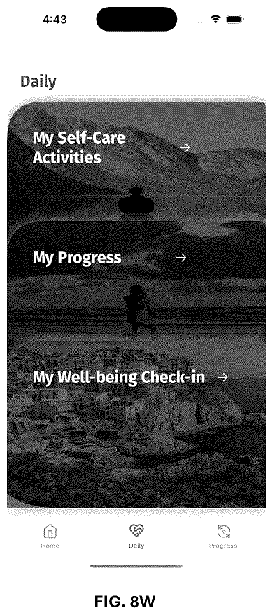

Next a Daily Survey Flow is detailed. When user begins the Daily Survey Flow, FIG. 8A will be displayed as an Introductory page. After clicking next, the user will be able to see questions and answer based on options in FIG. 8B.

Daily Survey Flow Questions

| Q. | Questions | Options |
|---|---|---|
| 1a | How are you feeling today? | Tired 0-10 Energetic |
| 1b | How are you feeling today? | Overwhelmed 0-10 Calm |
| 1c | How are you feeling today? | Insecure 0-10 Confident |
| 1d | How are you feeling today? | Ashamed 0-10 Proud |
| 1e | How are you feeling today? | Hot-tempered 0-10 Peaceful |
| 1f | How are you feeling today? | Lonely 0-10 Emotionally |
| | | Invested |
| 1g | How are you feeling today? | Bored 0-10 Amused |
| 1h | How are you feeling today? | Sad 0-10 Joyful |
| 1i | How are you feeling today? | Emotionally flooded 0-10 |
| | | Relieved |
| 1j | How are you feeling today? | Worried 0-10 Carefree |
| 1k | How are you feeling today? | Numb 0-10 Sensitive |
| 1l | How are you feeling today? | Discouraged 0-10 Enthusiast |

| Q | Questions | Description | Value |
|---|---|---|---|
| 2 | How many hours did you | Less than 2 | 0% |
| | sleep last night? | hours | |

| | | |
|---|---|---|
| 2-4 | hours | 33.33% |
| 4-6 | hours | 66.67% |
| 6-8 | hours | 100% |
| 8-10 | hours | 50% |
| 10+ | hours | 0% |

| | | | |
|---|---|---|---|
| 3 | How well did you sleep | Great | 100% |
| | last night? | Good | 75% |
| | | Ok | 50% |
| | | Bad | 25% |
| | | Awful | 0% |
| 4 | How many meals did you | 0 | 0% |
| | have yesterday? | 1 | 50% |
| | | 2 | 100% |
| | | 3 | 100% |
| | | 3+ | 0% |
| 5 | What did you eat yesterday? | Protein | |
| | | Vegetables | |
| | | Fruit | |
| | | Carbohydrate | |
| | | Processed food | |
| 6 | How many times did you eat | 0 | 0% |
| | protein yesterday? | 1 | 50% |
| | | 2 | 100% |
| | | 3 | 50% |
| | | 3+ | 0% |
| 7 | How many times did you eat | 0 | 0% |
| | vegetables yesterday? | 1 | 50% |
| | | 2 | 100% |
| | | 3 | 50% |
| | | 3+ | 0% |
| 8 | How many times did you eat | 0 | 0% |
| | fruit yesterday? | 1 | 50% |
| | | 2 | 100% |
| | | 3 | 50% |
| | | 3+ | 0% |
| 9 | How many times did you eat | 0 | 0% |
| | carbohydrates yesterday? | 1 | 50% |
| | | 2 | 100% |
| | | 3 | 50% |
| | | 3+ | 0% |
| 10 | How many times did you eat | 0 | 0% |
| | processed foods (chips, | 1 | 50% |
| | sweets, fast food) | 2 | 100% |
| | yesterday? | 3 | 50% |
| | | 3+ | 0% |
| 11 | What did you drink | Water | |
| | yesterday? | Juice | |
| | | Tea | |
| | | Coffee | |
| | | Soda | |
| | | Alcohol | |
| | | Milk | |
| 11a | How many glasses of Water | 0 | 0% |
| | you drank? | 1 | 0% |
| | | 2 | 0% |
| | | 3 | 50% |
| | | 3+ | 100% |
| 11b | How many glasses of Juice | 0 | 0% |
| | you drank? | 1 | 100% |
| | | 2 | 66.67% |
| | | 3 | 33.33% |
| | | 3+ | 0% |
| 11c | How many glasses of Tea | 0 | 80% |
| | you drank? | 1 | 100% |
| | | 2 | 50% |
| | | 3 | 25% |
| | | 3+ | 0% |
| 11d | How many glasses of Coffee | 0 | 80% |
| | you drank? | 1 | 100% |
| | | 2 | 50% |
| | | 3 | 25% |
| | | 3+ | 0% |
| 11e | How many glasses of Soda? | 0 | 100% |
| | | 1 | 80% |
| | | 2 | 0% |
| | | 3 | 0% |
| | | 3+ | 0% |
| 11f | How many glasses of Alcohol? | 0 | 100% |
| | | 1 | 80% |
| | | 2 | 0% |
| | | 3 | 0% |
| | | 3+ | 0% |
| 11g | How many glasses of Milk? | 0 | 0% |
| | | 1 | 33.33% |
| | | 2 | 66.67% |
| | | 3 | 100% |
| | | 3+ | 0% |
| 12 | How many times have you been | 0 | 0% |
| | sexually active in the | 1 | 0% |
| | past 7 days? | 2 | 100% |
| | | 3 | 0% |
| | | 4 | 0% |
| | | 5 | 0% |
| | | 6+ | 0% |
| 13 | How satisfied are you with | 1 | 0% |
| | your sex life? 1 = | 2 | 0% |
| | Very unsatisfied. | 3 | 0% |
| | 10 = Very satisfied. | 4 | 25% |
| | | 5 | 50% |
| | | 6 | 80% |
| | | 7 | 100% |
| | | 8 | 0% |
| | | 9 | 0% |
| | | 10 | 0% |
| 14 | Have you masturbated in the | Yes | 50% |
| | past 7 days? | No | 50% |
| 15 | Have you been sexually | Yes | 50% |
| | active with someone else in | No | 50% |
| | the past 7 days? | | |
| 16 | How many hours per day do you | Less than 1 | 100% |
| | usually spend exercising? | hour | |

| | | |
|---|---|---|
| 1-2 | hours | 100% |
| 2-3 | hours | 0% |
| 3-4 | hours | 0% |
| 4+ | hours | 0% |

| | | | |
|---|---|---|---|
| 17 | How intense is your typical | Very light | 0% |
| | workout routine? | Light | 50% |
| | | Moderate | 100% |
| | | High | 50% |
| | | Very high | 0% |
| 18 | What do you identify as? | Male | |
| | | Female | |
| | | Other | |

If Selected “Female” in Q18 the following questions are posed:

| | | | |
|---|---|---|---|
| Q | Question | Option | Value |

| | | |
|---|---|---|
| 19 | Are you currently menstruating? | Yes |
| | | No |
| 20 | What kinds of menstrual | Migraine |
| | discomfort do you usually | Stress |
| | experience? | Cramps |
| | | No discomfort |

| | | | |
|---|---|---|---|
| Q | Question | Option | Value |

| | | |
|---|---|---|
| 21 | Are you currently employed? | Yes |
| | | No |

If Employed then Following Questions are posed.

| Q | Question | Option | | Value |
|---|---|---|---|---|
| 22 | How many hours do you work a | Less than 8 hours | | 0% |
| | day? | 8 | hours | 100% |
| | | 9-10 | hours | 50% |
| | | 10+ | hours | 0% |

| | | |
|---|---|---|
| 23 | Is your job aligned with your | Yes |
| | values and life's purpose? | No |

| Q | Question | Option | | Value |
|---|---|---|---|---|
| 24 | How many hours did you spend | Less than 8 hours | | 100% |
| | yesterday on Social Media? | 8 | hours | 80% |
| | | 9-10 | hours | 50% |
| | | 10+ | hours | 0% |

After completion of answering all questions, FIG. 8C renders ‘You're all done!’. After continuing, the user can view her report under ‘Discover of Insights’ in FIG. 8D.

FIG. 8E depicts a Start of Daily Journey, if the user clicks on ‘Give it a go’, then she will be asked to select at least one self-care activity to begin which is in FIG. 8E. FIG. 8F shows the Start of Daily Journey, here as the Journey goes on from Day 1 to Day 7, the seeds will be transforming into a tree. After completing all 7 Days of Daily journey FIG. 8H will appear and provide the user with the option to share feedback or Discover connected self-care Activities. If the user selects Discover connected self-care activities the intro pages are shown in FIG. 8I and FIG. 8J. After the introductory pages of connected activities of FIGS. 8H-8I, a third introductory page is shown in FIG. 8K. After the third intro page connected activities can be selected including activities such as Dive shown in FIG. 8L, Practice shown in FIG. 8M, Reflection shown in FIG. 8N, and Self-Care introduction page in FIG. 8O which transitions to Day 1 Self Care of FIG. 8P through the Self Care Completion page of FIG. 8Q and the survey for Self-Care activity in FIG. 8R.

After completion of one activity, the system will prompt the user to select a new activity as shown in FIG. 8S—Selecting a new self-care activity. At the end of completing self-care activities at the end of day 7 the butterfly is rendered as emerging from a cocoon which means the user has been transformed as depicted in FIG. b8T—After completing self-care activities.

After completion of Self-care activities, the home page is shown where self-care activities history is shown to select from Reflection, Flash, Practice and Dive (FIG. 8U). The UI of FIG. 8U includes a Daily and progress option and if the user selects reflection then there is an option to see completed activities and started activities which is shown in FIG. 8V—activities started and completed. The Daily button when clicked provies options to show self-care activities, progress and well-being check-in as shown in FIG. 8W-Daily button.

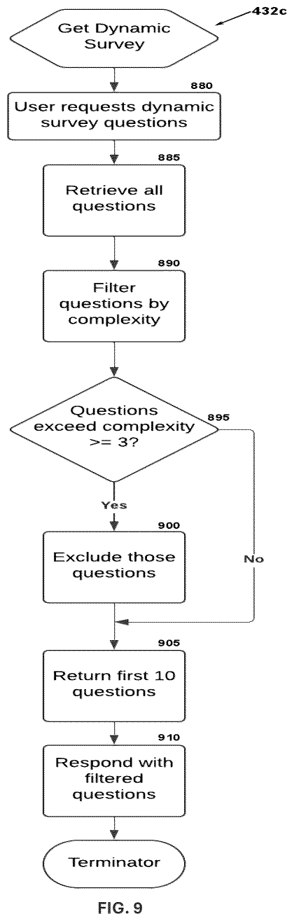

FIG. 9 shows an exemplary Dynamic Survey Get Flowchart. FIG. 9 starts when the user initiates a request for dynamic survey questions, beginning the process at Node 432c. In Step 880, the user submits a request to obtain a dynamic set of survey questions tailored to specific requirements in the table surveys_dailysurveyquestions. The system retrieves all available questions at Step 885, gathering the full list of possible questions for further filtering. In Step 890, the system applies a filtering process based on the complexity level of each question, preparing to deliver a set that matches the desired complexity.

At Step 895, the system checks whether any questions exceed a complexity level of 3. If there are questions that meet this criterion, the system proceeds to exclude those questions at Step 900, refining the list to ensure only questions within the acceptable complexity range are included. After filtering out overly complex questions, the system moves to Step 905, where it selects the first ten questions from the remaining set, preparing a limited, manageable number of questions for user response. Finally, in Step 910, the system responds to the user with this filtered set of questions, completing the dynamic survey retrieval process. The flow concludes after sending the filtered questions, marking the end of the Dynamic Survey Get process.

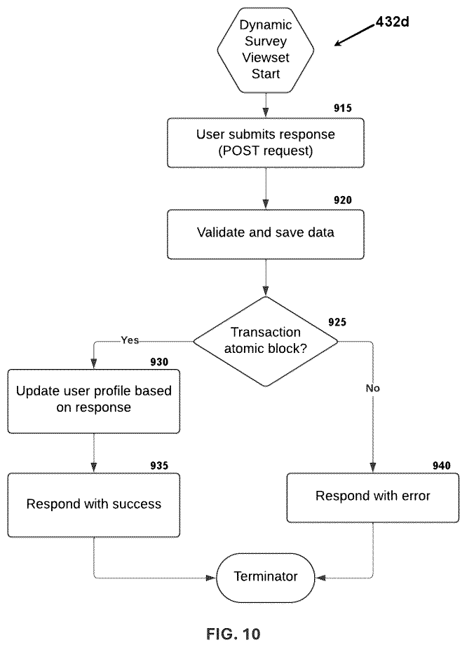

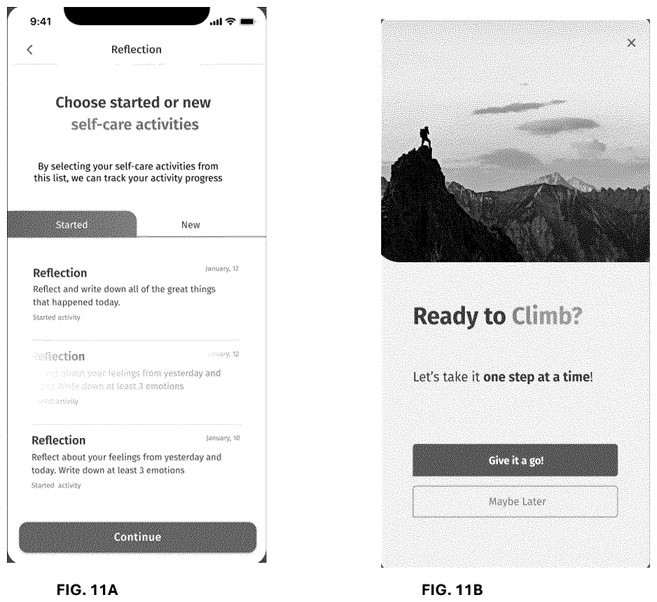

FIG. 10 shows an exemplary Create Dynamic Survey flowchart. The process begins when a user submits their response to a dynamic survey, initiating the process at Node 432d. In Step 915, the user sends their survey response through a POST request, which prompts the system to validate and save the data at Step 920 in the table surveys.dailysurveyresponse. During this step, the system checks for data integrity, ensuring that all required fields are correctly populated and that the response conforms to expected formats. Following the validation, the system enters a decision point at Step 925 to determine whether the transaction meets the requirements for an atomic block, which ensures that all database operations are executed as a single unit.

If the transaction is confirmed as an atomic block, the system proceeds to Step 930, where it updates the user's profile based on the response. This update incorporates insights from the survey response, adjusting the user profile to reflect new or modified information. Once the profile update is complete, the system responds to the user with a success message at Step 935, indicating that the response has been successfully processed and saved. If, however, the transaction does not qualify as an atomic block, the system diverts to Step 940, responding with an error to inform the user of an issue during processing. The flow concludes after sending either a success or error response, marking the end of the Create Dynamic Survey process.

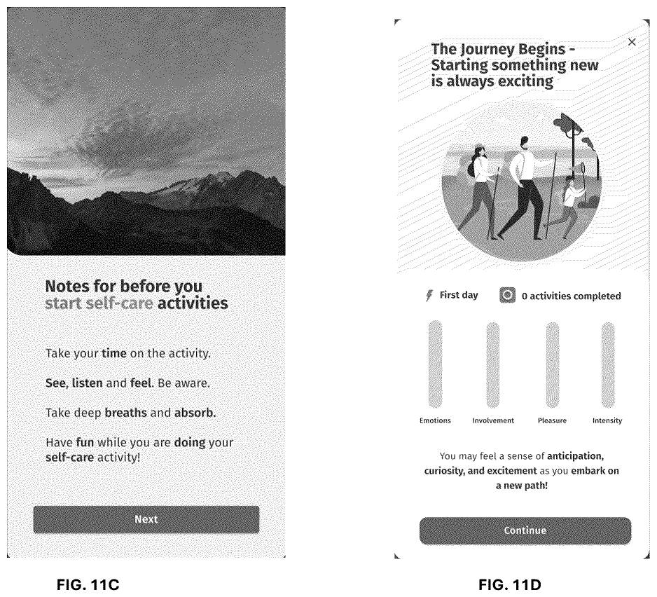

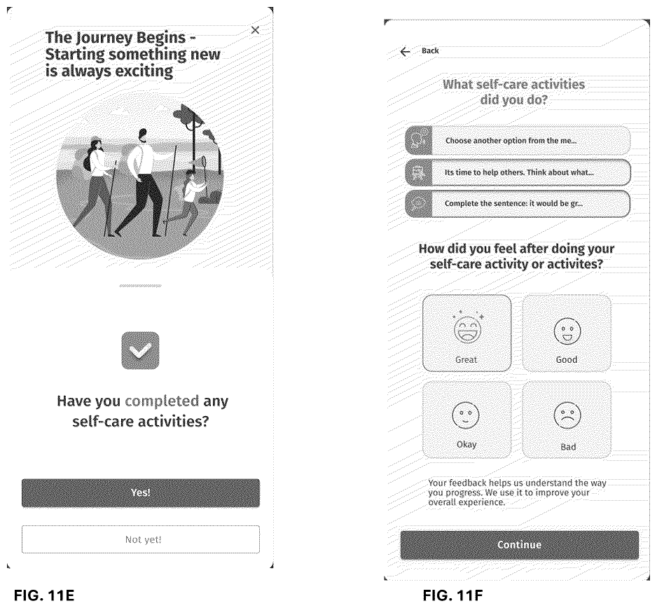

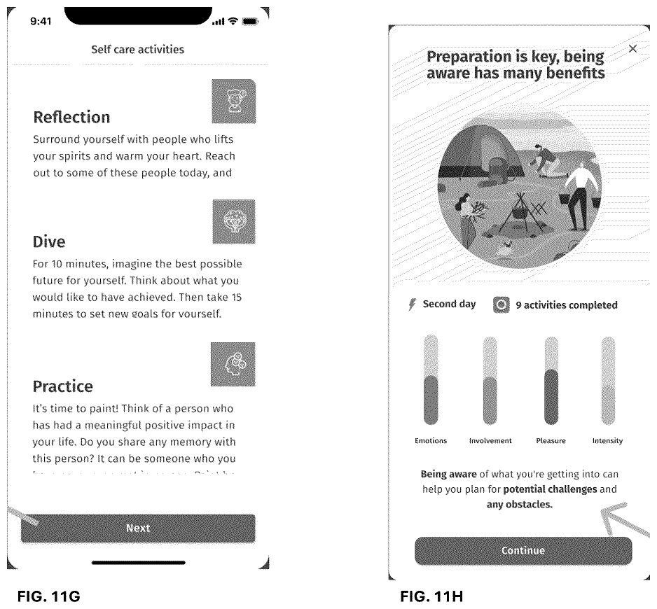

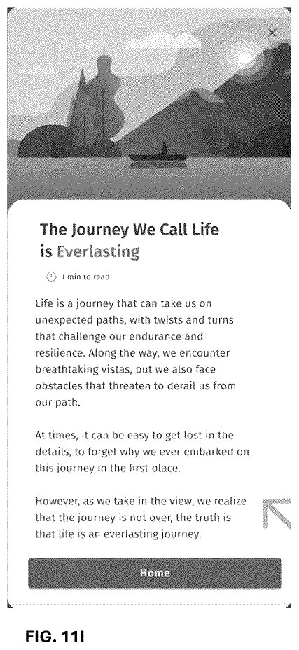

When the user selects My Self-care activities from FIG. 8W-Daily page, the user can choose started or new self-care activities page which is FIG. 11A-Self Care Activity started after which if the user selects on new and starts a self care activity then the intro page will be shown which is FIG. 11B-Self-care activity Introduction. After the self-care activity Intro1 user will be displayed Intro2 which is FIG. 11C-Self-care activity Intro2 after which the day wise self care activity is started with day 1 which is FIG. 11D-Self-care activity day 1.

At the end of the day the user should complete the self-care activity completed check to see whether the user completed the activities or not which is FIG. 11E—Self-care activity completed check. Users can not proceed to day 2 without completing Day 1. After the user completes the activity and selects Yes the survey will be shown to get the feedback of the user which is FIG. 11F—Self-care activity survey.

If the user has selected all the self-care activities and clicks continue it will show the next 3 new self-care activities depending on their report. The new self care activities list is FIG. 11G—Self-care activities list. On the next day till day 7, each day progress will be shown in UI in colorful vertical bars which is shown in FIG. 11H—Self-care activity the next day. On the last day of the self-care activity after completion, the final message will be shown to complete the self-care activity journey which is shown in FIG. 11I—Self-care activity completion.

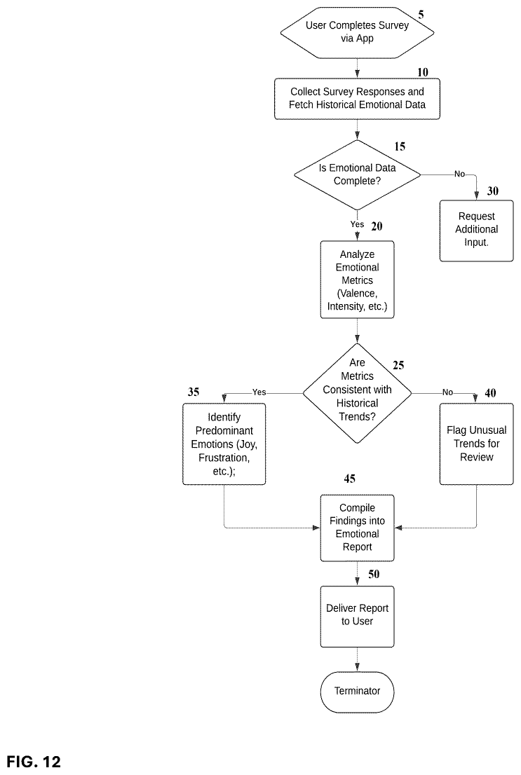

FIG. 12 shows an exemplary process to perform Emotional Survey: User Emotional Placement-Customized Emotion Management Report. This flowchart depicts the process of tracking and visualizing a user's emotional progress. The system begins by retrieving the user's historical emotional data (10). The process checks if the emotional data is complete (15) and if not requests additional input (30) and otherwise analyzes this data to identify trends and patterns over time (20). The process then checks if metrics are consistent with historical data (25) and if not flags unusual trends for review (40) and otherwise identifies one or more predominant emotions (joy, frustration, . . . ). Based on this analysis, the system generates visual representations of the user's emotional progress, such as graphs or charts (45). These visualizations are presented to the user in an easily understandable format (50). The user can interact with the visualizations, potentially setting new emotional goals or adjusting existing ones and the system updates the user's progress tracking information based on their interactions and new data.

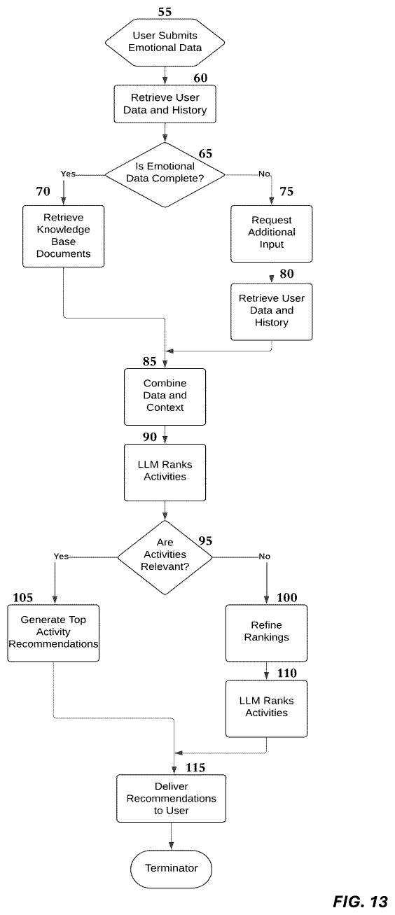

FIG. 13 shows an exemplary Activity Ranking Flowchart for Personalized Self-Care Recommendations. The user submits emotional data (55) and user data/history is retrieved (60). The process checks for completeness of data (65) and if sufficient retrieves the knowledge base documents (70) and otherwise requests additional data (75) and retrieves the user data and history (80). From 70 or 80, the process combines data and context (85) and applies the LLM to rank the activities for the user (90). The LLM output is checked for relevancy (95) and if relevant, the process generates top activity recommendations for the user (105) and otherwise refine the rankings (100) and request the LLM to rank the activities (110). From 105 or 110, the process delivers the recommendations to the user and exits.

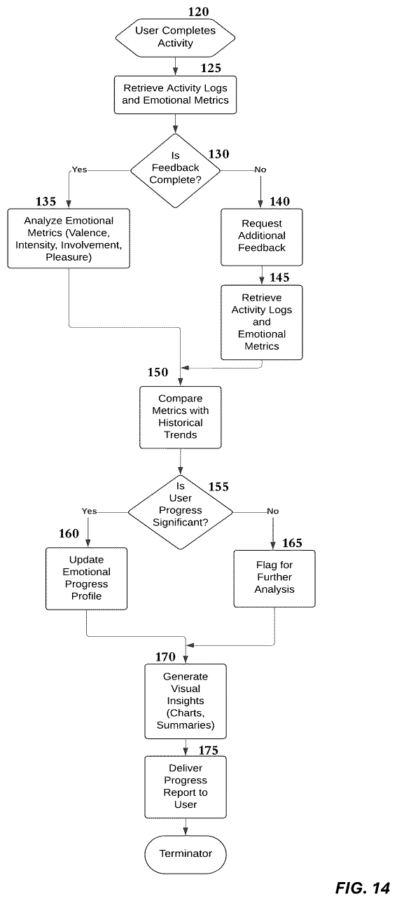

FIG. 14 shows an exemplary Progress Tracking Flowchart for Evaluating and Visualizing User Emotional Patterns. The process for monitoring and displaying a user's emotional progress over time starts by retrieving activity logs and emotional metrics (125). The process checks for completeness of feedback data (130). If complete, the process analyzes the emotional metrics such as valence, intensity, involvement, pleasure, among others at 135, and otherwise the process requests additional feedback data (140) and retrieves the activity logs and emotional metrics (145). From 135 or 145, the process analyzes Emotional Trends (150) where the collected data is then analyzed to identify patterns, trends, and changes in the user's emotional state over the specified time period. The process analyzes the sufficiency of user progress (155) and if progress is sufficient updates the emotional progress profile of the user. Otherwise the profile is flagged for further analysis or expert review (165). Based on the analysis, the system creates visual representations of the user's emotional progress (170). These may include graphs, charts, or other graphical elements that effectively communicate the identified trends. The result is presented to the user (175) and exits. The system displays the generated visualizations to the user in an easily comprehensible format, allowing them to see their emotional journey at a glance. This enables the user to interact with the visualizations. They can explore different aspects of their emotional data and, importantly, set new emotional goals or modify existing ones based on the insights gained. Finally, the system incorporates any new information, including the user's interactions and goal adjustments, into its database. This ensures that future progress tracking and visualizations will reflect the most current data and user preferences.

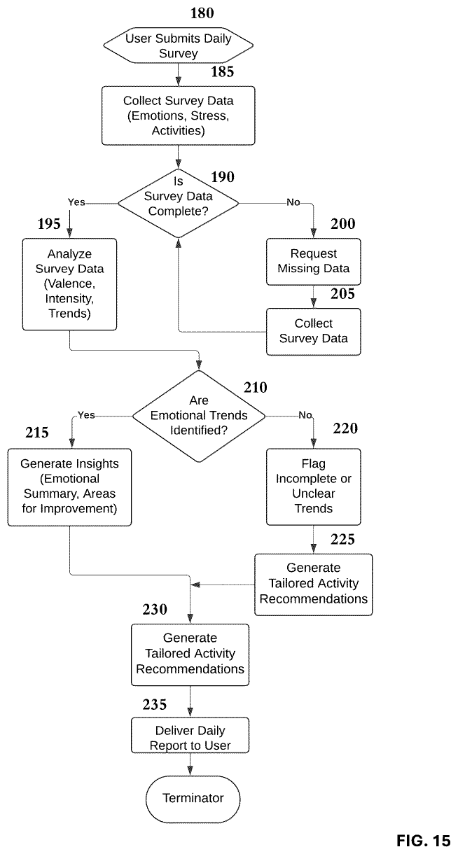

FIG. 15 shows an exemplary process to handle daily surveys. The process for monitoring and displaying a user's daily activities starts by retrieving activity logs and emotional metrics (185). The process checks for completeness of feedback data (190). If complete, the process analyzes the survey data such as valence, intensity, involvement, pleasure, among others at 195, and otherwise the process requests additional data (200) and retrieves the survey data (205). From 195 or 205, the process analyzes Emotional Trends (210) where the collected data is then analyzed to identify patterns, trends, and changes in the user's emotional state over the specified time period. The process generates insights such as emotional summary, areas for improvements, among others (215) and otherwise the profile is flagged for further analysis or expert review (220) and generates tailored activity recommendations (225). Based on the analysis, the system creates tailored activity recommendations (235). The result is presented to the user (235) and exits.

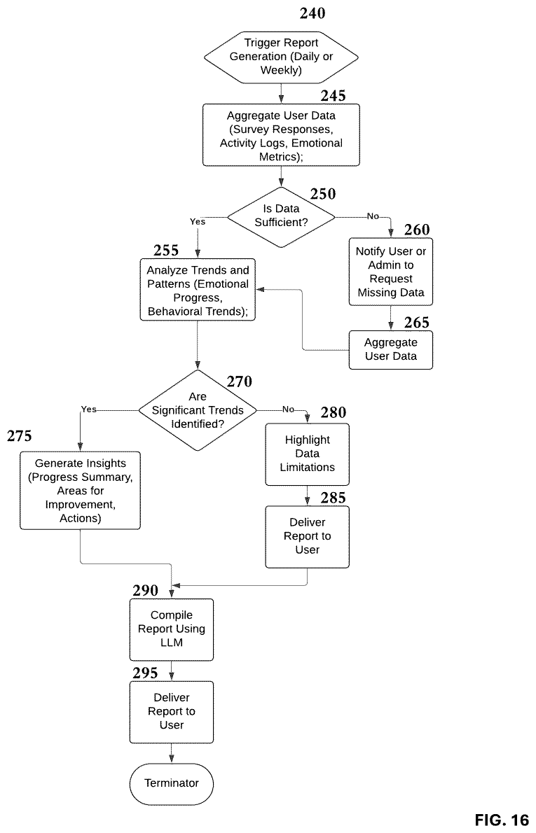

FIG. 16 shows an exemplary Insights Report Generation Flowchart for Aggregating, Analyzing, and Delivering Emotional Trends. The reported can be triggered on a periodic basis (240). The process aggregates user data such as survey responses, activity logs, and emotional metrics (245). The process checks for data sufficiency (250) and if sufficient it analyzes the trends and patterns such as emotional progress and behavior trends (255) and otherwise it requests missing data (260) and aggregates the new data (265) and continues with the analysis (255). When done, the process determines the presence of significant emotion trends (270) and if so, the process generates the insights including progress summary and areas for improvements/actions (275). If no new trend can be identified, the process highlights the data limitations (280) and delivers the report to the user (285). From 275 or 285, the process compiles a report using the LLM (290) and delivers the report to the user (295) and exits.

FIG. 12 shows in part the Emotional Survey process, which guides users through an assessment of their emotional state and generates a customized emotion management report. FIG. 13 outlines the Activity Ranking Flow, which provides personalized self-care recommendations based on the user's emotional data and activity history.

Pseudo code is disclosed below for FIG. 14's Progress Tracking Flow, showing how the system evaluates and visualizes the user's emotional progress over time, FIG. 15's Daily Survey process captures the user's daily emotional state and provides immediate feedback, and FIG. 16's Insights Report Generation Flow aggregates and analyzes emotional trends to deliver comprehensive insights to the user.

```
  Emotional Survey Flow
  function conductEmotionalSurvey(user):
    if user.hasCompletedSurveyBefore( ):
      return “Survey already completed”
    questions = fetchSurveyQuestions
    responses = presentQuestionsAndGetResponses(questions)
    if validateResponses(responses):
      saveToDatabase(“accounts_emotionalsurvey”, responses)
      report = generateEmotionalReport(responses)
      saveToDatabase(“accounts_emotionalreport”, report)
      return “Survey completed successfully”
    else:
      return “Validation errors in responses”
  Daily Survey
  function conductDailySurvey(user):
    if user.hasCompletedDailySurvey( ):
      return “Daily survey already completed”
      questions = fetchDailySurveyQuestions( )
    responses = presentQuestionsAndGetResponses(questions)
      if validateResponses(responses):
      saveToDatabase(“accounts_dailysurvey”, responses)
      insights = generateInsights(responses)
      saveToDatabase(“accounts_insights”, insights)
      return “Daily survey completed successfully”
    else:
      return “Validation errors in responses”
  Activity Ranking Flow
  function provideSelfCareRecommendations(user):
    emotionalState = getUserEmotionalState(user)
    surveyResponses = getUserSurveyResponses(user)
    recommendations =
    generateActivityRecommendations(emotionalState,
surveyResponses)
    rankedActivities = rankActivities(recommendations)
      return presentTopRankedActivities(rankedActivities)
  Progress Tracking Flow
  function trackUserProgress(user):
    data = collectUserData(user, [“dailySurveys”, “emotionalSurveys”,
“activityFeedback”])
      analysisResults = analyzeDataForTrends(data)
      visualizations = generateVisualizations(analysisResults)
      return presentProgressVisualizations(visualizations)
```

Together, these flowcharts represent a holistic approach to emotional management, combining data collection, analysis, personalized recommendations, and progress tracking to support users in understanding and improving their emotional well-being. The disclosed system and method represent a significant advancement in the field of artificial intelligence applied to mental health and well-being, delivering a dynamic, responsive, and deeply personalized service to users seeking to enhance their emotional intelligence and align their lives with their core values and aspirations.

1. A computer-implemented method for generating personalized emotional management, comprising:
receiving emotional state input from a user;
processing the emotional state vector for the user by:
aggregating the mapped numerical weights using a deterministic aggregation function, and
normalizing the aggregated values across a plurality of predefined emotional categories;
retrieving historical emotional state vectors and prior activity engagement data associated with the user from a database;
calculating one or more trend metrics by comparing the emotional state vector with the retrieved historical emotional state vectors using a predefined longitudinal analysis function;
generating personalized emotional management recommendations based on the processed emotional state input and retrieved information;
providing the personalized emotional management recommendations to the user; and
tracking user behavior and engagement with the provided recommendations.

2. The method of claim 1, further comprising sending a push notification updates and reminders to the user.

3. The method of claim 1, further comprising providing customized self-care activities to the user, wherein providing customized self-care activities comprises applying algorithms based on mathematical modeling and emotional circumplex analysis to generate and deliver individualized self-care activities tailored to the user's emotional state and track their progress to increase emotional intelligence.

4. The method of claim 1, further comprising generating a customized emotion management report for the user by placing them in one of the four quadrants based on the user's answers to one or more surveys and the emotions selected by software from the emotional complexity model.

5. The method of claim 1, further comprising tracking user progress in emotional management over time.

6. The method of claim 1, further comprising analyzing and managing the frequency and intensity of positive and negative emotions experienced by the user.

7. The method of claim 1, further comprising providing exercises to improve the user's self-consciousness and self-enhancement, wherein the exercises are implemented as structured activity modules stored in memory, each activity module being associated with a predefined emotional category threshold and wherein completion of an activity module triggers recalculation of the emotional state vector and dynamic re-ranking of remaining activity modules.

8. The method of claim 1, further comprising recommending activities to enhance the user's motivation and social skills, wherein the plurality of self-care activities are organized into predefined categories, and wherein the rule-based scoring model assigns different weights to the categories based on the user's emotional state and prior activity history to determine which activities are presented to the user.

9. The method of claim 1, further comprising implementing strategies to prevent mental illness by boosting positive emotions and reducing negative emotions.

10. The method of claim 1, further comprising providing guidance to help the user live aligned to their values and purpose.

11. The method of claim 1, comprising processing the user emotional state, customized self-care activities, activity progress tracking, a Retrieval-Augmented Generation (RAG) code and a custom Large Language Model (LLM).

12. The method of claim 1, wherein receiving the emotional state input from the user comprises receiving at least one of a textual description of the user's feelings, a voice input, or physiological signal data captured by one or more sensors, and converting the received input into the emotional state vector using a predefined feature extraction function.

13. The method of claim 1, wherein processing the emotional state vector for the user further comprises applying a multimodal fusion model configured to combine at least facial expression data, voice intonation data, and text-based sentiment data into the emotional state vector prior to aggregating the mapped numerical weights.

14. The method of claim 1, wherein the plurality of predefined emotional categories comprises at least one of joy, frustration, sadness, anger, fear, anxiety, and gratitude, and wherein the normalization distributes the aggregated values across said emotional categories to generate a normalized emotional profile for the user.

15. The method of claim 1, wherein calculating the one or more trend metrics comprises computing at least one of a moving average of emotional category scores, an intensity change rate, and an occurrence frequency of selected emotional categories over a specified time window to identify longitudinal emotional patterns for the user.

16. The method of claim 1, wherein generating personalized emotional management recommendations comprises selecting and ranking a set of self-care activities from an activity library based on the emotional state vector, the one or more trend metrics, and prior activity engagement data, using a trained recommendation model configured to output a ranked list of activities.

17. The method of claim 1, wherein providing the personalized emotional management recommendations to the user comprises generating a customized emotion management report including at least a visual representation of the normalized emotional profile, a summary of the one or more trend metrics, and a list of recommended activities with associated rationales.

18. The method of claim 1, wherein tracking user behavior and engagement with the provided recommendations comprises logging at least completion status, time of interaction, user feedback scores, and follow-up emotional state inputs for each recommendation, and updating the prior activity engagement data in the database based on the logged information.

19. The method of claim 1, further comprising dynamically updating future personalized emotional management recommendations by adapting at least one parameter of the deterministic aggregation function or the predefined longitudinal analysis function in response to the tracked user behavior and engagement, thereby personalizing subsequent recommendations for the user over time.

20. The method of claim 1, comprising:
receiving, via a user interface of a mobile device, a plurality of structured survey responses corresponding to predefined emotional and behavioral questions;
mapping each survey response to a predefined numerical weight stored in a scoring matrix;
ranking, using a rule-based scoring model, a plurality of predefined self-care activities, the ranking being based on:
the emotional state vector,
the one or more trend metrics, and
historical user engagement metrics including completion frequency and prior activity feedback;
generating, based on the ranking, a set of recommended self-care activities for presentation to the user;
updating the historical emotional state vectors and engagement metrics based on subsequent user interactions with the recommended self-care activities; and
generating a visual representation of emotional trends over time using the emotional state vectors and trend metrics.
